SEQUENCE LISTING (9 sequences)

SEQ ID NO: 1 - protein, 5 aa, Homo sapiens
GYNWH

SEQ ID NO: 2 - protein, 16 aa, Homo sapiens
EITHSGSTNYNPSLKS

SEQ ID NO: 3 - protein, 14 aa, Homo sapiens
EIAVAGTGYYGMDV

SEQ ID NO: 4 - protein, 11 aa, Homo sapiens
RASQGINSAFA

SEQ ID NO: 5 - protein, 7 aa, Homo sapiens
DASSLES

SEQ ID NO: 6 - protein, 9 aa, Homo sapiens
QQFNSYPLT

SEQ ID NO: 7 - protein, 448 aa, Homo sapiens
QVQLQQWGAGLLKPSETLSLTCAVYGGSFSGYNWHWIRQPPGKGLEWIGEITHSGSTNYNPSLKSRVTISVDTSKNQFSLKLSSVTAADTAVYYCVREIAVAGTGYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 8 - protein, 214 aa, Homo sapiens
AIQLTQSPSSLSASVGDRVTITCRASQGINSAFAWYQQKPGKAPKLLIYDASSLESGVPSRFSGSGSGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLTQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMV

(19) United States
(12) Patent Application Publication
(10) Pub. No.: US 2013/0315913 A1
(43) Pub. Date: Nov. 28, 2013

(54) ANTI-LIGHT ANTIBODY THERAPY FOR INFLAMMATORY BOWEL DISEASE

(71) Applicant: SANOFI, (US)

(72) Inventor: Meng ZHANG, Bridgewater, NJ (US)

(73) Assignee: SANOFI, Paris (FR)

(21) Appl. No.: 13/840,260

(22) Filed: Mar. 15, 2013

Related U.S. Application Data
(60) Provisional application No. 61/615,601, filed on Mar. 26, 2012.

(30) Foreign Application Priority Data
Feb. 6, 2013 (FR) 1351012

(51) Int. Cl. C07K 16/24; G01N 33/68; A61K 49/00

(52) U.S. Cl. CPC C07K 16/24; A61K 49/0004; G01N 33/6854
USPC 424/139.1; 530/387.9; 424/9.2; 435/7.92

(57) ABSTRACT

The present invention provides safe therapeutic doses of an antagonist of human LIGHT (lymphotoxin-like, exhibits inducible expression and competes with Herpes Virus Glycoprotein D for Herpes Virus Entry Mediator (HVEM), a receptor expressed by T lymphocytes), as well as methods of monitoring whether a therapeutic dose of an anti-LIGHT antagonist is safe.

### RELATED APPLICATIONS

This application claims priority to U.S. Provisional Application No. 61/615,601 filed Mar. 26, 2012, and French Patent Application No. 1351012 filed Feb. 6, 2013, the content of which are hereby incorporated herein in its entirety.

### FIELD OF THE INVENTION

The present invention relates to the field of therapeutic treatment for inflammatory bowel disease. More specifically, the invention relates to safe doses of anti-LIGHT antagonists, such as anti-LIGHT antibodies.

### BACKGROUND

Inflammatory bowel disease (IBD), which affects an estimated 600,000 Americans per year, is a group of inflammatory conditions of the colon and small intestine. The major types of IBD are Crohn's disease (CD) and ulcerative colitis (UC). Accounting for far fewer cases are other forms of IBD, such as: collagenous colitis, lymphocytic colitis, ischemic colitis, diversion colitis, Behçet's disease, and indeterminate colitis.

The main difference between CD and UC is the location and nature of the inflammatory changes. CD can affect any part of the gastrointestinal tract, from mouth to anus (skip lesions), although a majority of the cases start in the terminal ileum. UC, in contrast, is restricted to the colon and the rectum. In addition, CD and UC present with extra-intestinal manifestations, such as liver problems, arthritis, skin manifestations, and eye problems, in different proportions.

Although very different diseases, both CD and UC may present with any of the following symptoms: abdominal pain, vomiting, diarrhea, rectal bleeding, severe internal cramps/muscle spasms in the region of the pelvis, weight loss, and various associated complaints or diseases such as arthritis, pyoderma gangrenosum, and primary sclerosing cholangitis. Diagnosis is generally by colonoscopy with biopsy of pathological lesions. While IBD can limit a person's quality of life because of pain, vomiting, diarrhea, and other socially unacceptable symptoms, it is rarely fatal on its own. Fatalities due to complications, such as toxic megacolon, bowel perforation, and surgical complications, are also rare. Despite this, the treatment of IBD places considerable demands on the healthcare system and is associated with increased healthcare costs.

Optimal treatment of IBD depends on its form. For example, mesalazine is more useful in UC than in CD. Generally, depending on the level of severity, IBD may require immunosuppression with drugs, such as prednisone, TNF inhibition, azathioprine (Imuran), methotrexate, or 6-mercaptopurine. More commonly, treatment of IBD requires a form of mesalazine. Often, steroids are used to control disease flares. Recently, biologicals have been used, such as TNF inhibitors. Severe cases may require surgery, such as bowel resection, strictureplasty, or a temporary or permanent colostomy or ileostomy.

Usually treatment is started by administering drugs with high anti-inflammatory effects, such as prednisone. Once the inflammation is successfully controlled, the patient is usually switched to a lighter drug to keep the disease in remission, such as Asacol, a mesalazine. If unsuccessful, a combination of the aforementioned immunosuppression drugs with a mesalazine (which may also have an anti-inflammatory effect) may or may not be administered, depending on the patient. The goal of treatment is toward achieving remission, after which the patient is usually switched to a lighter drug with fewer potential side effects. Every so often, however, an acute resurgence of the original symptoms may appear; which is known as a “flare-up”. Depending on the circumstances, it may go away on its own or require medication. The time between flare-ups may be anywhere from weeks to years, and varies wildly between patients and therapies.

Therefore, there is a pressing need in the art for improved treatment regimens that are more consistently effective in treating IBD, and which are non-immunogenic and safe for use in humans.

### SUMMARY OF THE INVENTION

The present disclosure improves upon current treatment options by providing safe therapeutic doses of an antagonist of human LIGHT (lymphotoxin-like, exhibits inducible expression and competes with Herpes Virus Glycoprotein D for Herpes Virus Entry Mediator (HVEM), a receptor expressed by T lymphocytes), as well as methods of monitoring whether a therapeutic dose of an anti-LIGHT antagonist is safe.

An embodiment of the invention is a maximal safe therapeutic dose of a LIGHT antagonist having an area under the plasma concentration versus time curve calculated using the trapezoidal method from time zero to real time (AUCₗₐₛₜ) from about 100 mg·day/L to about 6000 mg·day/L following administration to a human subject. In some embodiments, the AUCₗₐₛₜ is selected from the group consisting of about 100, 250, 500, 750, 1000, 1500, 2000, 2500, 3000, 3500, 4000, 4500, 5000, 5500, and 6000 mg·day/L. In some embodiments, the AUCₗₐₛₜ is selected from the group consisting of about 193, 642, 1710, 2430, 4630, and 6000 mg·day/L.

An embodiment of the invention is a maximal safe therapeutic dose of a LIGHT antagonist having an area under the plasma concentration versus time curve extrapolated to infinity (AUC) from about 150 mg·day/L to about 6000 mg·day/L following administration to a human subject. In some embodiments, the AUC is selected from the group consisting of about 100, 150, 250, 500, 750, 1000, 1500, 2000, 2500, 3000, 3500, 4000, 4500, 5000, 5500, and 6000 mg·day/L. In some embodiments, the AUC is selected from the group consisting of about 207, 698, 2060, and 4600 mg·day/L.

An embodiment of the invention is a maximal safe therapeutic dose of a LIGHT antagonist having a maximum plasma concentration observed (Cₘₐₓ) from about 3.5 mg/L to about 175 mg/L following administration to a human subject. In some embodiments, the Cₘₐₓ is selected from the group consisting of about 3.5, 5, 10, 20, 30, 40, 50, 60, 70, 80, 90, 100, 110, 120, 130, 140, 150, 160, 170, and 175 mg/L. In some embodiments, the Cₘₐₓ is selected from the group consisting of about 5.27, 17.9, 45.7, 122, and 147 mg/L.

An embodiment of the invention is a maximal safe therapeutic dose of a LIGHT antagonist having a first time to reach a maximum plasma concentration (tₘₐₓ) from about 4 days to about 9 days following administration to a human subject. In some embodiments, the tₘₐₓ is selected from the group consisting of about 4, 4.5, 5, 5.5, 6, 6.5, 7, 7.5, 8, 8.5, and 9 days. In some embodiments, the tₘₐₓ is selected from the group consisting of about 5, 6.75, 7, and 8.5 days.

An embodiment of the invention is a maximal safe therapeutic dose of a LIGHT antagonist having a time to reach terminal half life (t_{1/2}^{Z}) from about 3 days to about 47 days following administration of a single dose to a human subject. In some embodiments, the tᵥ₂^{z} is selected from the group consisting of about, 3, 5, 7, 9, 11, 13, 15, 17, 19, 21, 23, 25, 27, 29, 31, 33, 35, 37, 39, 41, 43, 45, and 47 days. In some embodiments, the t_{1/2}^{z} is selected from the group consisting of about 13.6, 18.7, 21.3, 28.9, and 32 days.

In certain embodiments the invention provides safe therapeutic doses of a LIGHT antagonist which is an antibody or antigen-binding fragment that specifically binds LIGHT. In certain embodiments, the antibody or antigen-binding fragment comprises heavy and light chain CDR sequences from the HCVR/LCVR sequence pair of SEQ ID NOs: 10/11. In one embodiment, the antibody or antigen-binding fragment comprises three heavy chain complementarity determining region (HCDR) sequences comprising SEQ ID NOs: 1, 2, and 3, respectively, and three light chain complementarity determining (LCDR) sequences comprising SEQ ID NOs: 4, 5, and 6, respectively. In another embodiment, the antibody or antigen-binding fragment comprises an HCVR having the amino acid sequence of SEQ ID NO: 10 and an LCVR having the amino acid sequence of SEQ ID NO: 11.

In certain embodiments the invention, the safe therapeutic dose of the LIGHT antagonist is a dose of an anti-LIGHT antibody equal to or less than about 1200 mg. In certain embodiments, the therapeutic dose is selected from the group consisting of about 40 mg, 120 mg, 300 mg, 600 mg, 900 mg and 1200 mg. In certain embodiments, the therapeutic dose is about 1200 mg.

One embodiment of the invention provides a method of monitoring whether a therapeutic dose of a LIGHT antagonist administered to a human subject is safe, said method comprising: (a) administering said therapeutic dose of said LIGHT antagonist to said human subject; (b) measuring one or more events selected from the group consisting of: intensive treatment in an emergency room or at home for allergic bronchospasm, blood dyscrasias, or convulsions; development of drug dependency or drug abuse; alanine aminotransferase (AL T)>3× upper limit of normal range (ULN) associated with total bilirubin >2×ULN or ALT increase >2×ULN; diagnosis of cancer during study; QTc≧=500 ms; systemic hypersensitivity reactions or anaphylaxis; severe injection site reaction; infection including opportunistic infections; pregnancy; and overdose, and (c) determining one or more said events as measured in (b) has occurred, wherein said therapeutic dose is identified as not safe and the therapeutic dose is discontinued or lowered.

Another embodiment of the invention is a method of monitoring whether a therapeutic dose of a LIGHT antagonist administered to a human subject is safe, said method comprising: (a) administering said therapeutic dose of said LIGHT antagonist to said human subject; (b) measuring one or more events selected from the group consisting of: intensive treatment in an emergency room or at home for allergic bronchospasm, blood dyscrasias, or convulsions; development of drug dependency or drug abuse; alanine aminotransferase (AL T)>3× upper limit of normal range (ULN) associated with total bilirubin >2×ULN or ALT increase >2×ULN; diagnosis of cancer during study; QTc≧=500 ms; systemic hypersensitivity reactions or anaphylaxis; severe injection site reaction; infection including opportunistic infections; pregnancy; and overdose, and (c) determining one or more said events as measured in (b) has not occurred, wherein said therapeutic dose is identified as said safe therapeutic dose having been administered to said human subject.

In certain embodiments, the method of monitoring includes measuring an infection. In some embodiments, the infection is an upper respiratory tract infection.

In certain embodiments, the method of monitoring includes measuring an injection site reaction. In some embodiments, the injection site reaction is erythema, pain, or edema. In some embodiments, the erythema diameter is ≧9 mM. In some embodiments, the edema diameter is ≧9 mM. In some embodiments, the pain is assessed as at least mild using the present pain intensity (PPI) assessment.

An embodiment of the invention is a method of quantifying or monitoring an amount of anti-drug antibodies in blood serum of a human subject following administration of drug wherein the drug is a LIGHT antagonist, said method comprising: (a) administering a dose of said LIGHT antagonist to said human subject; (b) obtaining a sample of said blood serum from said human subject; and (b) determining the amount of anti-drug antibodies in said serum sample.

In certain embodiments the invention provides methods which include administering a therapeutic dose of a LIGHT antagonist which is an antibody or antigen-binding fragment that specifically binds LIGHT. In certain embodiments, the antibody or antigen-binding fragment comprises heavy and light chain CDR sequences from the HCVR/LCVR sequence pair of SEQ ID NOs: 10/11. In one embodiment, the antibody or antigen-binding fragment comprises three heavy chain complementarity determining region (HCDR) sequences comprising SEQ ID NOs: 1, 2, and 3, respectively, and three light chain complementarity determining (LCDR) sequences comprising SEQ ID NOs: 4, 5, and 6, respectively. In another embodiment, the antibody or antigen-binding fragment comprises an HCVR having the amino acid sequence of SEQ ID NO: 10 and an LCVR having the amino acid sequence of SEQ ID NO: 11.

In certain embodiments the invention provides methods which include administering a therapeutic dose of a LIGHT antagonist which is a dose of an anti-LIGHT antibody equal to or less than about 1200 mg. In certain embodiments, this therapeutic dose is selected from the group consisting of about 40 mg, 120 mg, 300 mg, 600 mg, 900 mg and 1200 mg. In certain embodiments, the therapeutic dose is about 1200 mg.

One embodiment of the invention is a pharmaceutical composition comprising a safe therapeutic dose of a LIGHT antagonist together with one or more physiologically acceptable excipients.

One embodiment of the invention provides a method for treating an inflammatory bowel disease (IBD) in a subject in need thereof comprising administering to the subject a pharmaceutical composition which comprises a safe therapeutic dose of a LIGHT antagonist together with one or more physiologically acceptable excipients. The LIGHT antagonist is an anti-LIGHT antibody that is administered at a dose of at least 40 mg to about 1200, thereby treating the IBD in the subject. The anti-LIGHT antibody is administered at a dose of about 1200 mg in some embodiments. In some embodiments, the pharmaceutical composition comprising an anti-LIGHT antibody is administered as a single dose. In some embodiments, the antibody is administered by injection, including subcutaneously.

One embodiment of the invention provides a kit comprising the pharmaceutical composition, which comprises a safe therapeutic dose of a LIGHT antagonist together with one or more physiologically acceptable excipient, and a container. In some embodiments, the LIGHT antagonist is an antibody or antigen-binding fragment that specifically binds LIGHT. The kit can further comprise a label. In some embodiments, the label contains reference to one or more adverse reactions or side effects in connection with the administration of the LIGHT antagonist. The adverse reactions or side effects are selected from the group consisting of: intensive treatment in an emergency room or at home for allergic bronchospasm, blood dyscrasias, or convulsions; development of drug dependency or drug abuse; alanine aminotransferase (AL T)>3× upper limit of normal range (ULN) associated with total bilirubin >2×ULN or ALT increase >2×ULN; diagnosis of cancer during study; QTc≧=500 ms; systemic hypersensitivity reactions or anaphylaxis; severe injection site reaction; infection including opportunistic infections; pregnancy; and overdose. In some embodiments, the injection site reactions are pain, erythema, or edema. In some embodiments, the label indicates that the adverse reactions or side effects can primarily occur in higher dose levels, such as about 500 mg or more per injection.

### DETAILED DESCRIPTION OF THE INVENTION

### A. Definitions

Unless defined otherwise, all technical and scientific terms used herein have the same meaning as is commonly understood by one of ordinary skill in the art.

It is noted here that as used in this specification and the appended claims, the singular forms “a”, “an”, and “the” also include plural reference, unless the context clearly dictates otherwise.

The term “about” or “approximately” means within 10%, and more preferably within 5% (or 1% or less) of a given value or range.

The terms “administer” or “administration” refer to the act of injecting or otherwise physically delivering a substance as it exists outside the body (e.g., a formulation of the invention) into a patient, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

In the context of a polypeptide, the term “analog” refers to a polypeptide that possesses a similar or identical function as a LIGHT polypeptide, a fragment of a LIGHT polypeptide, a LIGHT epitope, or an anti-LIGHT antibody, but does not necessarily comprise a similar or identical amino acid sequence of a LIGHT polypeptide, a fragment of a LIGHT polypeptide, a LIGHT epitope, or an anti-LIGHT antibody, or possess a similar or identical structure of a LIGHT polypeptide, a fragment of a LIGHT polypeptide, a LIGHT epitope, or an anti-LIGHT antibody. A polypeptide that has a similar amino acid sequence refers to a polypeptide that satisfies at least one of the following: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, and preferably at least 90%, more preferably at least 95%, or most preferably at least 99% identical to the amino acid sequence of a LIGHT polypeptide (e.g., SEQ ID NO: 9), a fragment of a LIGHT polypeptide, a LIGHT epitope, or an anti-LIGHT antibody described herein; (b) a polypeptide encoded by a nucleotide sequence that hybridizes under stringent conditions to a nucleotide sequence encoding a LIGHT polypeptide, a fragment of a LIGHT polypeptide, a LIGHT epitope, or an anti-LIGHT antibody (or VH or VL region thereof) described herein of at least 5 amino acid residues, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues (see, e.g., Sambrook et al. (2001) Molecular Cloning: A Laboratory Manual, Cold Spring Harbor Laboratory Press, Cold Spring Harbor, N.Y.; Maniatis et al. (1982) Molecular Cloning: A Laboratory Manual, Cold Spring Harbor Press, Cold Spring Harbor, N.Y.); and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, and preferably at least 90%, more preferably at least 95%, or most preferably at least 99% identical to the nucleotide sequence encoding a LIGHT polypeptide, a fragment of a LIGHT polypeptide, a LIGHT epitope, or an anti-LIGHT antibody (or VH or VL region thereof) described herein. A polypeptide with similar structure to a LIGHT polypeptide, a fragment of a LIGHT polypeptide, a LIGHT epitope, or an anti-LIGHT antibody refers to a polypeptide that has a similar secondary, tertiary or quaternary structure of a LIGHT polypeptide, a fragment of a LIGHT, a LIGHT epitope, or a LIGHT antibody. The structure of a polypeptide can determined by methods known to those skilled in the art, including but not limited to, X-ray crystallography, nuclear magnetic resonance, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences (e.g., amino acid sequences or nucleic acid sequences) can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to nucleic acid molecules of interest. BLAST protein searches can be performed with the XBLAST program parameters set, e.g., to score 50, wordlength=3 to obtain amino acid sequences homologous to a protein molecule of interest. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web at ncbi dot nlm dot nih dot gov). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

An “antagonist” or “inhibitor” of LIGHT refers to a molecule that is capable of inhibiting or otherwise decreasing one or more of the biological activities of LIGHT, such as in a cell expressing LIGHT or in a cell expressing a LIGHT ligand, such as a LIGHT receptor. For example, in certain embodiments, antibodies of the invention are antagonist antibodies that inhibit or otherwise decrease secretion of CCL20, IL-8, and/or RANTES from a cell having a cell surface-expressed LIGHT receptor (e.g., HVEM, LTβR and/or DcR3) when said antibody is contacted with said cell. In some embodiments, an antagonist of LIGHT (e.g., an antagonistic antibody of the invention) may, for example, act by inhibiting or otherwise decreasing the activation and/or cell signaling pathways of the cell expressing a LIGHT receptor, thereby inhibiting a LIGHT-mediated biological activity of the cell relative to the LIGHT-mediated biological activity in the absence of antagonist. In certain embodiments of the invention, the anti-LIGHT antibodies are fully human, antagonistic anti-LIGHT antibodies, preferably fully human, monoclonal, antagonistic anti-LIGHT antibodies.

The terms “antibody”, “immunoglobulin”, or “Ig” may be used interchangeably herein. The term antibody includes, but is not limited to, synthetic antibodies, monoclonal antibodies, recombinantly produced antibodies, multispecific antibodies (including bi-specific antibodies), human antibodies, humanized antibodies, chimeric antibodies, intrabodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies, and epitope-binding fragments of any of the above. In particular, antibodies include immunoglobulin molecules and immunologically active portions of immunoglobulin molecules, i.e., antigen binding domains or molecules that contain an antigen-binding site that specifically binds to a LIGHT antigen (e.g., one or more complementarity determining regions (CDRs) of an anti-LIGHT antibody). The anti-LIGHT antibodies can be of any type (e.g., IgG, IgE, IgM, IgD, IgA and IgY), any class (e.g., IgG1, IgG2, IgG3, IgG4, IgA1 and IgA2), or any subclass (e.g., IgG2a and IgG2b) of immunoglobulin molecule. In preferred embodiments, the anti-LIGHT antibodies are fully human, such as fully human monoclonal anti-LIGHT antibodies. In certain embodiments, the anti-LIGHT antibodies are IgG antibodies, human IgG4 antibodies.

The terms “composition” and “formulation” are intended to encompass a product containing the specified ingredients (e.g., an anti-LIGHT antibody) in, optionally, the specified amounts, as well as any product which results, directly or indirectly, from the combination of the specified ingredients in, optionally, the specified amounts.

The terms “constant region” or “constant domain” refer to a carboxy terminal portion of the light and heavy chain which is not directly involved in binding of the antibody to antigen but exhibits various effector functions, such as interaction with the Fc receptor. The terms refer to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CHL domain of the light chain.

The term “epitope” refers to a localized region on the surface of an antigen, such as a LIGHT polypeptide or LIGHT polypeptide fragment, that is capable of being bound to one or more antigen binding regions of an antibody, and that has antigenic or immunogenic activity in an animal, preferably a mammal, and most preferably in a human, that is capable of eliciting an immune response. An epitope having immunogenic activity is a portion of a polypeptide that elicits an antibody response in an animal. An epitope having antigenic activity is a portion of a polypeptide to which an antibody specifically binds, as determined by any method well known in the art, for example, such as an immunoassay. Antigenic epitopes need not necessarily be immunogenic. Epitopes usually consist of chemically active surface groupings of molecules, such as amino acids or sugar side chains, and have specific three dimensional structural characteristics, as well as specific charge characteristics. A region of a polypeptide contributing to an epitope may be contiguous amino acids of the polypeptide or the epitope may come together from two or more non-contiguous regions of the polypeptide. The epitope may or may not be a three-dimensional surface feature of the antigen. In certain embodiments, a LIGHT epitope is a three-dimensional surface feature of a LIGHT polypeptide (e.g., in a trimeric form of a LIGHT polypeptide). In other embodiments, a LIGHT epitope is a linear feature of a LIGHT polypeptide (e.g., in a trimeric form or monomeric form of the LIGHT polypeptide). Anti-LIGHT antibodies may specifically bind to an epitope of the monomeric (denatured) form of LIGHT, an epitope of the trimeric (native) form of LIGHT, or both the monomeric (denatured) form and the trimeric (native) form of LIGHT. In specific embodiments, the anti-LIGHT antibodies specifically bind to an epitope of the trimeric form of LIGHT but do not specifically bind the monomeric form of LIGHT.

The term “excipients” refers to inert substances that are commonly used as a diluent, vehicle, preservative, binder, stabilizing agent, etc. for drugs and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, Remington's Pharmaceutical Sciences (1990) Mack Publishing Co., Easton, Pa., which is hereby incorporated by reference in its entirety.

In the context of a peptide or polypeptide, the term “fragment” refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence. Fragments may, for example, result from alternative RNA splicing or from in vivo protease activity. In certain embodiments, LIGHT fragments include polypeptides comprising an amino acid sequence of at least 50, at 100 amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a LIGHT polypeptide. In a specific embodiment, a fragment of a LIGHT polypeptide or an antibody that specifically binds to a LIGHT antigen retains at least 1, at least 2, or at least 3 functions of the full-length polypeptide or antibody.

The terms “fully human antibody” or “human antibody” are used interchangeably herein and refer to an antibody that comprises a human variable region and, most preferably a human constant region. In specific embodiments, the terms refer to an antibody that comprises a variable region and constant region of human origin. “Fully human” anti-LIGHT antibodies, in certain embodiments, can also encompass antibodies that bind LIGHT polypeptides and are encoded by nucleic acid sequences that are naturally occurring somatic variants of a human germline immunoglobulin nucleic acid sequence. In a specific embodiment, the anti-LIGHT antibodies are fully human antibodies. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). Methods of producing fully human antibodies are known in the art.

The phrase “recombinant human antibody” includes human antibodies that are prepared, expressed, created, or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al. (1992) Nucl. Acids Res. 20:6287-6295) or antibodies prepared, expressed, created, or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “heavy chain” when used in reference to an antibody refers to five distinct types, called alpha (α), delta (Δ), epsilon (ε), gamma (γ) and mu (μ), based on the amino acid sequence of the heavy chain constant domain. These distinct types of heavy chains are well known in the art and give rise to five classes of antibodies, IgA, IgD, IgE, IgG, and IgM, respectively, including four subclasses of IgG, namely IgG1, IgG1, IgG3, and IgG4. Preferably the heavy chain is a human heavy chain.

An “isolated” or “purified” antibody is substantially free of cellular material or other contaminating proteins from the cell or tissue source from which the antibody is derived, or substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, or 5% (by dry weight) of heterologous protein (also referred to herein as a “contaminating protein”). When the antibody is recombinantly produced, it is also preferably substantially free of culture medium, i.e., culture medium represents less than about 20%, 10%, or 5% of the volume of the protein preparation. When the antibody is produced by chemical synthesis, it is preferably substantially free of chemical precursors or other chemicals, i.e., it is separated from chemical precursors or other chemicals that are involved in the synthesis of the protein. Accordingly, such preparations of the antibody have less than about 30%, 20%, 10%, 5% (by dry weight) of chemical precursors or compounds other than the antibody of interest. In a preferred embodiment, anti-LIGHT integrin antibodies are isolated or purified.

The terms “Kabat numbering,” and like terms are recognized in the art and refer to a system of numbering amino acid residues that are more variable (i.e. hypervariable) than other amino acid residues in the heavy and light chain variable regions of an antibody, or an antigen binding portion thereof (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242). For the heavy chain variable region, the hypervariable region typically ranges from amino acid positions 31 to 35 for CDR1, amino acid positions 50 to 65 for CDR2, and amino acid positions 95 to 102 for CDR3. For the light chain variable region, the hypervariable region typically ranges from amino acid positions 24 to 34 for CDR1, amino acid positions 50 to 56 for CDR2, and amino acid positions 89 to 97 for CDR3.

The term “light chain” when used in reference to an antibody refers to two distinct types, called kappa (κ) of lambda (λ) based on the amino acid sequence of the constant domains. Light chain amino acid sequences are well known in the art. In preferred embodiments, the light chain is a human light chain.

The terms “manage”, “managing”, and “management” refer to the beneficial effects that a subject derives from a therapy (e.g., a prophylactic or therapeutic agent), which does not result in a cure of the infection. In certain embodiments, a subject is administered one or more therapies (e.g., prophylactic or therapeutic agents) to “manage” a LIGHT-mediated disease (e.g., IBD), one or more symptoms thereof, so as to prevent the progression or worsening of the disease.

The term “monoclonal antibody” refers to an antibody obtained from a population of homogenous or substantially homogeneous antibodies, and each monoclonal antibody will typically recognize a single epitope on the antigen. In preferred embodiments, a “monoclonal antibody” is an antibody produced by a single hybridoma or other cell. The term “monoclonal” is not limited to any particular method for making the antibody. For example, monoclonal antibodies may be made by the hybridoma method as described in Kohler et al.; Nature, 256:495 (1975) or may be isolated from phage libraries. Other methods for the preparation of clonal cell lines and of monoclonal antibodies expressed thereby are well known in the art (see, for example, Chapter 11 in: Short Protocols in Molecular Biology, (2002) 5th Ed.; Ausubel et al., eds., John Wiley and Sons, New York).

The term “pharmaceutically acceptable” means being approved by a regulatory agency of the Federal or a state government, or listed in the U.S. Pharmacopeia, European Pharmacopeia or other generally recognized Pharmacopeia for use in animals, and more particularly in humans.

By “pharmaceutically acceptable excipient” is meant any inert substance that is combined with an active molecule, such as a monoclonal antibody, for preparing an agreeable or convenient dosage form. The “pharmaceutically acceptable excipient” is an excipient that is non-toxic to recipients at the dosages and concentrations employed, and is compatible with other ingredients of the formulation comprising the monoclonal antibody.

The terms “prevent”, “preventing”, and “prevention” refer to the total or partial inhibition of the development, recurrence, onset or spread of a LIGHT-mediated disease and/or symptom related thereto, resulting from the administration of a therapy or combination of therapies provided herein (e.g., a combination of prophylactic or therapeutic agents).

The term “LIGHT antigen” refers to that portion of a LIGHT polypeptide to which an antibody specifically binds. A LIGHT antigen also refers to an analog or derivative of a LIGHT polypeptide or fragment thereof to which an antibody specifically binds. In some embodiments, a LIGHT antigen is a monomeric LIGHT antigen or a trimeric LIGHT antigen. A region of a LIGHT polypeptide contributing to an epitope may be contiguous amino acids of the polypeptide, or the epitope may come together from two or more non-contiguous regions of the polypeptide. The epitope may or may not be a three-dimensional surface feature of the antigen. A localized region on the surface of a LIGHT antigen that is capable of eliciting an immune response is a LIGHT epitope. The epitope may or may not be a three-dimensional surface feature of the antigen.

The term “human LIGHT,” “hLIGHT” or “hLIGHT polypeptide” and similar terms refer to the polypeptides (“polypeptides,” “peptides” and “proteins” are used interchangeably herein) comprising the amino acid sequence of SEQ ID NO: 9 and related polypeptides, including SNP variants thereof. Related polypeptides include allelic variants (e.g., SNP variants); splice variants; fragments; derivatives; substitution, deletion, and insertion variants; fusion polypeptides; and interspecies homologs, preferably, which retain LIGHT activity and/or are sufficient to generate an anti-LIGHT immune response. Also encompassed are soluble forms of LIGHT that are sufficient to generate an anti-LIGHT immunological response. As those skilled in the art will appreciate, an anti-LIGHT antibody can bind to a LIGHT polypeptide, polypeptide fragment, antigen, and/or epitope, as an epitope is part of the larger antigen, which is part of the larger polypeptide fragment, which, in turn, is part of the larger polypeptide. hLIGHT can exist in a trimeric (native) or monomeric (denatured) form.

The terms “LIGHT-mediated disease” and “LIGHT-mediated disorder” are used interchangeably and refer to any disease that is completely or partially caused by or is the result of LIGHT, e.g., hLIGHT. In certain embodiments, LIGHT is aberrantly (e.g., highly) expressed on the surface of a cell. In some embodiments, LIGHT may be aberrantly upregulated on a particular cell type. In other embodiments, normal, aberrant, or excessive cell signaling is caused by binding of LIGHT to a LIGHT ligand. In certain embodiments, the LIGHT ligand is a LIGHT receptor (e.g., HVEM, LTβR, or DCR3), for example, that is expressed on the surface of a cell, such as a colonic epithelial cell. In certain embodiments, the LIGHT-mediated disease is an inflammatory bowel disease (IBD), such as Crohn's disease (CD) or ulcerative colitis (UC).

The terms “specifically binds” or “specifically binding” mean specifically binding to an antigen or a fragment thereof (e.g., LIGHT) and not specifically binding to other antigens. An antibody that specifically binds to an antigen may bind to other peptides or polypeptides with lower affinity, as determined by, e.g., radioimmunoassays (RIA), enzyme-linked immunosorbent assays (ELISA), BIACORE, or other assays known in the art. In certain embodiments, an anti-LIGHT antibody of the invention may specifically bind to LIGHT (e.g., hLIGHT) with more than two-fold greater affinity that a different, non-LIGHT antigen. Antibodies or variants or fragments thereof that specifically bind to an antigen may be cross-reactive with related antigens. For example, in certain embodiments an anti-LIGHT antibody may cross-react with hLIGHT and another LIGHT antigen (e.g., a rodent or non-human primate LIGHT antibody). Preferably, antibodies or variants or fragments thereof that specifically bind to an antigen do not cross-react with other non-LIGHT antigens. An antibody or a variant or a fragment thereof that specifically binds to a LIGHT antigen can be identified, for example, by immunoassays, BIAcore, or other techniques known to those of skill in the art. Typically a specific or selective reaction will be at least twice background signal or noise, and more typically more than 10 times background. See, e.g., Paul, ed., 1989, Fundamental Immunology Second Edition, Raven Press, New York at pages 332-336 for a discussion regarding antibody specificity.

The terms “subject” and “patient” are used interchangeably. As used herein, a subject is preferably a mammal, such as a non-primate (e.g., cows, pigs, horses, cats, dogs, rats, etc.) or a primate (e.g., monkey and human), most preferably a human. In one embodiment, the subject is a mammal, preferably a human, having a LIGHT-mediated disease. In another embodiment, the subject is a mammal, preferably a human, at risk of developing a LIGHT-mediated disease.

The term “therapeutic agent” refers to any agent that can be used in the treatment, management or amelioration of a LIGHT-mediated disease and/or a symptom related thereto. In certain embodiments, the term “therapeutic agent” refers to a LIGHT antibody of the invention. In certain other embodiments, the term “therapeutic agent” refers to an agent other than a LIGHT antibody of the invention. Preferably, a therapeutic agent is an agent that is known to be useful for, or has been or is currently being used for the treatment, management or amelioration of a LIGHT-mediated disease or one or more symptoms related thereto.

The term “therapeutic dose” refers to any agent(s) or dose of any agent(s) which can be used in the treatment, management or amelioration of a disease, disorder, malady and the like associated a LIGHT-mediated disease and/or a symptom related thereto.

The term “safe therapeutic dose” refers to the quantity of any agent(s) which can be used in the treatment, management or amelioration of a disease, disorder, malady and the like associated a LIGHT-mediated disease and/or a symptom related thereto while maintaining a clinically acceptable benefit/risk profile. A safe therapeutic dose is selected form the group consisting of 40 mg, 120 mg, 300 mg, 600 mg, 900 mg, and 1200 mg. An embodiment of a safe therapeutic dose is about 1200 mg or less than 1200 mg.

As used herein, the phrase “therapeutically effective amount” means a dose of LIGHT antagonist that results in a detectable improvement in one or more symptoms associated with inflammatory bowel disease (IBD) or which causes a biological effect (e.g., a decrease in the level of a particular biomarker) that is correlated with the underlying pathologic mechanism(s) giving rise to the condition or symptom(s) of inflammatory bowel disease. For example, a dose of LIGHT antagonist that causes an improvement in any of the following symptoms or conditions is deemed a “therapeutically effective amount”: abdominal pain, vomiting, diarrhea, rectal bleeding, severe internal cramps/muscle spasms in the region of the pelvis, weight loss, and various associated complaints or diseases such as arthritis, pyoderma gangrenosum, and primary sclerosing cholangitis.

An embodiment of the invention is identifying or monitoring a safe therapeutic dose of a LIGHT antagonist by measuring one or more events selected from the group consisting of: intensive treatment in an emergency room or at home for allergic bronchospasm, blood dyscrasias, or convulsions; development of drug dependency or drug abuse; alanine aminotransferase (AL T)>3× upper limit of normal range (ULN) associated with total bilirubin >2×ULN or ALT increase >2×ULN; diagnosis of cancer during study; QTc≧=500 ms; systemic hypersensitivity reactions or anaphylaxis; severe injection site reaction; infection including opportunistic infections; pregnancy; and overdose. The methods used to calculate the afore-mentioned events are discussed in detail in the examples presented below. Methods used to calculate the afore-mentioned events are commonly known to those skilled in the art.

The term “therapy” refers to any protocol, method, and/or agent that can be used in the prevention, management, treatment, and/or amelioration of a LIGHT-mediated disease (e.g., IBD). In certain embodiments, the terms “therapies” and “therapy” refer to a biological therapy, supportive therapy, and/or other therapies useful in the prevention, management, treatment, and/or amelioration of a LIGHT-mediated disease known to one of skill in the art, such as medical personnel.

The terms “treat”, “treatment”, and “treating” refer to the reduction or amelioration of the progression, severity, and/or duration of a LIGHT-mediated disease (e.g., IBD) resulting from the administration of one or more therapies (including, but not limited to, the administration of one or more prophylactic or therapeutic agents). In specific embodiments, such terms refer to the reduction or inhibition of the binding of LIGHT to HVEM, the reduction or inhibition of the binding of LIGHT to LTβR, the reduction or inhibition of the binding of LIGHT to DcR3, the reduction or inhibition of the production or secretion of CCL20 from a cell expressing a LIGHT receptor of a subject, the reduction or inhibition of the production or secretion of IL-8 from a cell expressing a LIGHT receptor of a subject, the reduction or inhibition of the production or secretion of RANTES from a cell expressing a LIGHT receptor of a subject, and/or the inhibition or reduction of one or more symptoms associated with a LIGHT-mediated disease such as IBD.

The terms “variable region” or “variable domain” refer to a portion of the light and heavy chains, typically about the amino-terminal 120 to 130 amino acids in the heavy chain and about 100 to 110 amino acids in the light chain, which differ extensively in sequence among antibodies and are used in the binding and specificity of each particular antibody for its particular antigen. The variability in sequence is concentrated in those regions called complementarity determining regions (CDRs), while the more highly conserved regions in the variable domain are called framework regions (FR). The CDRs of the light and heavy chains are primarily responsible for the interaction of the antibody with antigen. Numbering of amino acid positions is according to the EU Index, as in Kabat et al. (1991) Sequences of proteins of immunological interest. (U.S. Department of Health and Human Services, Washington D.C.) 5^{th} ed. a (“Kabat et al.”). In preferred embodiments, the variable region is a human variable region.

An embodiment of the invention is detection methods to measure total human antibody levels or the proportion of a specific antibody (for example, a LIGHT antibody) or to measure anti-drug antibodies in a test sample. The test sample can be any bodily sample from a mammal. Non-limiting examples include blood samples, serum samples, and tissue samples. Detection methods may involve using a “capture device” in which one or more antibodies are attached to the capture device. Nonlimiting examples of capture devices include wells of a plate wherein the plate may include any number of wells such as a 12 well plate or a 96 well plate. However, capture devices are not limited to plates but may include any substrate that an antibody may attach to, for example, an elution column. An embodiment of the invention utilizes tab-labeled antibodies. The tag can be any tag capable of detection. Nonlimiting examples include florescent tags such as Rhoda mine, enzymatic tags such as luciferase or sulfa-tags.

### B. LIGHT

Lymphotoxin-like, exhibits inducible expression and competes with Herpes Virus Glycoprotein D for Herpes Virus Entry Mediator (HVEM), a receptor expressed by T lymphocytes (LIGHT) is a member of the Tumor Necrosis Factor (TNF) Superfamily (TNFSF14). LIGHT is also known as TNFSF14 or CD258. LIGHT may be purified from any natural source, or may be produced synthetically (e.g., by use of recombinant DNA technology).

Preferably, the LIGHT molecule is from a human, known herein as “hLIGHT”. hLIGHT has the following amino acid sequence, which is identified as SEQ ID NO: 9:

(SEQ ID NO: 9)

| | |
|---|---|
| 1 | MEESVVRPSV FVVDGQTDIP FTRLGRSHRR QSCSVARVGL |
| | GLLLLLMGAG |
| 51 | LAVQGWFLLQ LHWRLGEMVT RLPDGPAGSW EQLTQERRSH |
| | EVNPAAHLTG |
| 101 | ANSSLTGSGG PLLWETQLGL AFLRGLSYHD GALVVTKAGY |
| | YYIYSKVQLG |
| 150 | GVGCPLGLAS TITHGLYKRT PRYPEELELL VSQQSPCGRA |
| | TSSSRVWWDS |
| 200 | SFLGGVVHLE AGEEVVVRVL DERLVRLRDG TRSYFGAFMV. |

LIGHT binds to 3 distinct receptors, Herpes Virus Entry Mediator (HVEM), Lymphotoxin-β Receptor (LTβR), and decoy receptor 3 (DcR3), which exist in humans but not in mice. LIGHT expression is restricted to natural killer (NK) cells, monocytes, immature dendritic cells (DC), and activated T cells, where expression is inducible and transient in nature.

Membrane-bound LIGHT is susceptible to proteolysis cleavage and can be released in a soluble form. A novel, non-glycosylated LIGHT lacking the trans membrane domain (ΔTM) exists in the cell cytosol as a result of alternative RNA splicing. The alternately spliced isoform of LIGHT mRNA that encodes a trans membrane-deleted form (ΔTM) is detected in in vitro activated T cells and gives rise to a non-glycosylated protein. Platelets release a soluble LIGHT upon activation in vitro. This platelet-derived soluble LIGHT protein is biologically active and can induce an inflammatory response in monocytes and particularly within endothelial cells, measured as up-regulation of adhesion molecules and release of chemokine's. While membrane LIGHT disrupts the binding and inhibitory signaling between HVEM and B and T lymphocyte attenuator (BTLA), binding of soluble LIGHT enhances the binding of BTLA with HVEM. Thus, soluble LIGHT may be a key factor in determining whether HVEM and BTLA can interact and the generation of an inhibitory signal. Moreover, platelet-associated LIGHT is involved in adhesion of platelets to endothelial cells in vitro and it is co-localized in thrombus material removed from the site of plaque rupture in patients with ST elevation myocardial infarction (STEMI) undergoing percutaneous coronary intervention.

LIGHT interacts with LTβR on stromal cells, monocytes, dendritic cells (DC)s, and endothelial cells, and triggers various proinflammatory activities, including the induction of cytokines, chemokines, and adhesion molecules. In addition, LIGHT expression on T cells promotes their activation and longevity. The binding of membrane LIGHT with HVEM provides a co-stimulatory signal while the binding of HVEM to B-and T-lymphocyte attenuator delivers a co-inhibitory signal.

Inflammatory bowel disease (IBD), which encompasses Crohn's disease (CD) and ulcerative colitis (UC), is a chronic inflammatory disorder characterized by deregulated immune responses in predisposed individuals. The rationale for targeting LIGHT in IBD comes from both animal studies and human expression data. LIGHT is normally transiently expressed and highly regulated. Constitutive T cell expression of either human or mouse LIGHT leads to the development of a profound multi-organ autoimmune phenotype characterized by lymphadenopathy, auto-antibodies and cellular infiltrates into various peripheral tissues, especially the lamina propria and submucosa of the intestine. Adoptive transfer of mesenteric lymph node cells from the LIGHT transgenic mice into RAG−/− mice resulted in the development of a disease with key pathological features and cytokine profiles characteristic of CD. Additionally, inhibition of LIGHT/LTβα signaling by LTβR-Fc fusion protein alleviated intestinal inflammation in 3 mouse models of colitis. The concept that LIGHT provides a critical proinflammatory signal during cellular immune responses is reinforced by studies in IBD patients. LIGHT mRNA is up regulated in biopsies from inflamed areas of small bowel. Cell surface LIGHT is expressed on human mucosal T cells and NK cells but not on peripheral naïve T cells.

### C. LIGHT Antagonists

The present disclosure includes methods that comprise administering to a subject a LIGHT antagonist. The LIGHT antagonist may be any molecule, such as an antibody, a siRNA, a nucleic acid, an aptamer, a protein, or a small molecule organic compound.

In certain embodiments, the formulations of the invention include an anti-LIGHT antibody, or a variant thereof, or an antigen binding fragment thereof. Anti-LIGHT antibodies specifically bind to a LIGHT (lymphotoxin-like, exhibits inducible expression and competes with HSV glycoprotein D for HVEM, a receptor expressed by T lymphocytes) protein, polypeptide fragment, or epitope. The LIGHT molecule may be from any species.

In certain exemplary embodiments, the anti-LIGHT antibody is a humanized antibody, a fully human antibody, or a variant thereof or an antigen-binding fragment thereof. Preferred anti-LIGHT antibodies prevent binding of LIGHT with its receptors and inhibit LIGHT biological activity (e.g., the LIGHT-mediated production or secretion of CCL20, IL-8, or RANTES from cells expressing a LIGHT ligand, such as a LIGHT receptor (e.g., HVEM, LTβR, and/or DcR3).

In certain specific embodiments, the anti-LIGHT antibody comprises a heavy chain variable region (VH) comprising the amino acid sequence of any one or more of the following complementary determining regions (CDRs):

| | | |
|---|---|---|
| HCDR1 - | GYNWH; | (SEQ ID NO: 1) |
| HCDR2 - | EITHSGSTNYNPSLKS; | (SEQ ID NO: 2) |
| or | | |
| HCDR3 - | EIAVAGTGYYGMDV. | (SEQ ID NO: 3) |

In other specific embodiments, the anti-LIGHT antibody comprises a light chain variable region (VL) comprising the amino acid sequence of any one or more of the following complementary determining regions (CDRs):

| | | |
|---|---|---|
| LCDR1 - | RASQGINSAFA; | (SEQ ID NO: 4) |
| LCDR2 - | DASSLES; | (SEQ ID NO: 5) |
| or | | |
| LCDR3 - | QQFNSYPLT. | (SEQ ID NO: 6) |

In one specific embodiment, the anti-LIGHT antibody comprises a heavy chain variable region (VH) comprising the amino acid sequences of SEQ ID NOs: 1, 2, and 3.

In another specific embodiment, the anti-LIGHT antibody comprises a light chain variable region (VL) comprising the amino acid sequences of SEQ ID NOs: 4, 5, and 6.

In more specific embodiments, the anti-LIGHT antibody comprises a heavy chain variable region comprising the amino acid sequences of SEQ ID NOs: 1, 2, and 3; and a light chain variable region comprising the amino acid sequences of SEQ ID NOs: 4, 5, and 6.

In specific embodiments, the anti-LIGHT antibody comprises a heavy chain comprising the amino acid sequence of SEQ ID NO: 7:

(SEQ ID NO: 7)

| | |
|---|---|
| 1 | QVQLQQWGAG LLKPSETLSL TCAVYGGSFS GYNWHWIRQP |
| | PGKGLEWIGE |
| 51 | ITHSGSTNYN PSLKSRVTIS VDTSKNQFSL KLSSVTAADT |
| | AVYYCVREIA |
| 101 | VAGTGYYGMD VWGQGTTVTV SSASTKGPSV FPLAPCSRST |
| | SESTAALGCL |
| 151 | VKDYFPEPVT VSWNSGALTS GVHTFPAVLQ SSGLYSLSSV |
| | VTVPSSSLGT |
| 201 | KTYTCNVDHK PSNTKVDKRV ESKYGPPCPP CPAPEFEGGP |
| | SVFLFPPKPK |
| 251 | DTLMISRTPE VTCVVVDVSQ EDPEVQFNWY VDGVEVHNAK |
| | TKPREEQFNS |
| 301 | TYRVVSVLTV LHQDWLNGKE YKCKVSNKGL PSSIEKTISK |
| | AKGQPREPQV |
| 351 | YTLPPSQEEM TKNQVSLTCL VKGFYPSDIA VEWESNGQPE |
| | NNYKTTPPVL |
| 401 | DSDGSFFLYS RLTVDKSRWQ EGNVFSCSVM HEALHNHYTQ |
| | KSLSLSLG |

Positions 1-122: variable region of the heavy chain (VH). The CDRs (complementary determining regions, according to Kabat definition) are underlined.
Positions 123-448: constant region of human IgG4 (SwissProt IGHG4_HUMAN with the two mutations S241P and L248E, according to Kabat numbering).

In other specific embodiments, the anti-LIGHT antibody comprises a light chain comprising the amino acid sequence of SEQ ID NO: 8:

(SEQ ID NO: 8)

| | |
|---|---|
| 1 | AIQLTQSPSS LSASVGDRVT ITCRASQGIN SAFAWYQQKP |
| | GKAPKLLIYD |
| 51 | ASSLESGVPS RFSGSGSGTD FTLTISSLQP EDFATYYCQQ |
| | FNSYPLTFGG |
| 101 | GTKVEIKRTV AAPSVFIFPP SDEQLKSGTA SVVCLLNNFY |
| | PREAKVQWKV |
| 151 | DNALQSGNSQ ESVTEQDSKD STYSLSSTLT LSKADYEKHK |
| | VYACEVTHQG |
| 201 | LSSPVTKSFN RGEC |

Positions 1-107: variable region of the light chain (VL). The CDRs (complementary determining regions, according to Kabat definition) are underlined.
Positions 108-214: constant region of human CK.

In further embodiments, the anti-LIGHT antibody comprises a heavy chain comprising the amino acid sequence of SEQ ID NO: 7, and a light chain comprising the amino acid sequence of SEQ ID NO: 8.

In an exemplary embodiment of the invention, the anti-LIGHT antibody is a fully human antibody. Examples of fully human antibody isotypes include IgA, IgD, IgE, IgG, and IgM. Preferably, the anti-LIGHT antibody is an IgG antibody. There are four forms of IgG. Preferably, the anti-LIGHT antibody is an IgG4 antibody. In one embodiment of the invention, the anti-LIGHT antibody is a fully human IgG4 antibody.

In some embodiments, the anti-LIGHT antibody further comprises a constant region, e.g., a human IgG constant region. In some embodiments, the constant region is a human IgG4 constant region. In additional embodiments, the constant region is a modified human IgG4 constant region. In other embodiments, the constant region is a human CK constant region.

In a most preferred embodiment of the invention, the anti-LIGHT antibody is a fully human IgG4 anti-LIGHT antibody comprising a heavy chain comprising the amino acid sequence of SEQ ID NO: 7 and a light chain comprising the amino acid sequence of SEQ ID NO: 8 (referred to herein as the “F19 Antibody”). In an alternative most preferred embodiment of the invention, the anti-LIGHT antibody is a fully human IgG4 anti-LIGHT antibody comprising a heavy chain variable region and a light chain variable region, the heavy chain variable region comprising 3 complementary determining regions (CDRs) comprising the amino acid sequences of SEQ ID NOs: 1, 2, and 3, and the light chain variable region comprising 3 CDRs comprising the amino acid sequences of SEQ ID NOs: 4, 5, and 6. Identification, isolation, preparation, and characterization of anti-LIGHT antibodies, including the anti-LIGHT antibody comprising a heavy chain amino acid sequence comprising SEQ ID NO: 7 and a light chain amino acid sequence comprising SEQ ID NO: 8, have been described in detail in U.S. Pat. No. 8,058,402, corresponding to PCT Publication WO 2008/027338, which are incorporated herein by reference.

Certain embodiments of the invention also include variants of anti-LIGHT antibodies. Specifically, the invention may include variants of the anti-LIGHT antibody that is a fully human IgG4 anti-LIGHT antibody comprising a heavy chain comprising the amino acid sequence of SEQ ID NO: 7 and a light chain comprising the amino acid sequence of SEQ ID NO: 8. Variants of anti-LIGHT antibodies may have similar physicochemical properties based on their high similarity, and therefore are also included within the scope of the invention. Variants are defined as antibodies with an amino acid sequence that is at least 95%, preferably at least 97%, for instance at least 98% or 99% homologous to an anti-LIGHT antibody, and capable of competing for binding to a LIGHT polypeptide, a LIGHT polypeptide fragment, or a LIGHT epitope. Preferably, the variants will ameliorate, neutralize, or otherwise inhibit binding of LIGHT with its receptors and LIGHT biological activity (e.g., the LIGHT-mediated production or secretion of CCL20, IL-8, or RANTES from cells expressing a LIGHT ligand, such as a LIGHT receptor (e.g., HVEM, LTβR, and/or DcR3). Determining competition for binding to the target can be done by routine methods known to the skilled person in the art. Preferably the variants are human antibodies, and preferably are IgG4 molecules. In preferred embodiments, a variant is at least 95%, 96%, 97%, 98%, or 99% identical in amino acid sequence with the F19 Antibody. The term “variant” refers to an antibody that comprises an amino acid sequence that is altered by one or more amino acids compared to the amino acid sequences of the anti-LIGHT antibody. The variant may have conservative sequence modifications, including amino acid substitutions, modifications, additions, and deletions.

Examples of modifications include, but are not limited to, glycosylation, acetylation, pegylation, phosphorylation, amidation, derivatization by known protecting/blocking groups, proteolytic cleavage, and linkage to a cellular ligand or other protein. Amino acid modifications can be introduced by standard techniques known in the art, such as site-directed mutagenesis, molecular cloning, oligonucleotide-directed mutagenesis, and random PCR-mediated mutagenesis in the nucleic acid encoding the antibodies. Conservative amino acid substitutions include the ones in which the amino acid residue is replaced with an amino acid residue having similar structural or chemical properties. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine, tryptophan), nonpolar side chains (e.g., glycine, alanine, valine, leucine, isoleucine, proline, phenylalanine, methionine), beta-branched side chains (e.g., threonine, valine, isoleucine), and aromatic side chains (e.g., tyrosine, phenylalanine, tryptophan). It will be clear to the skilled artisan that classifications of amino acid residue families other than the one used above can also be employed. Furthermore, a variant may have non-conservative amino acid substitutions, e.g., replacement of an amino acid with an amino acid residue having different structural or chemical properties. Similar minor variations may also include amino acid deletions or insertions, or both. Guidance in determining which amino acid residues may be substituted, modified, inserted, or deleted without abolishing immunological activity may be found using computer programs well known in the art. Computer algorithms, such as, inter alia, Gap or Bestfit, which are known to a person skilled in the art, can be used to optimally align amino acid sequences to be compared and to define similar or identical amino acid residues. Variants may have the same or different, either higher or lower, binding affinities compared to an anti-LIGHT antibody, but are still capable of specifically binding to LIGHT, and may have the same, higher or lower, biological activity as the anti-LIGHT antibody.

Embodiments of the invention also include antigen binding fragments of the anti-LIGHT antibodies. The term “antigen binding domain,” “antigen binding region,” “antigen binding fragment,” and similar terms refer to that portion of an antibody that comprises the amino acid residues that interact with an antigen and confer on the binding agent its specificity and affinity for the antigen (e.g., the complementary determining regions (CDR)). The antigen binding region can be derived from any animal species, such as rodents (e.g., rabbit, rat or hamster) and humans. Preferably, the antigen binding region will be of human origin. Non-limiting examples of antigen binding fragments include: Fab fragments, F(ab′)2 fragments, Fd fragments, Fv fragments, single chain Fv (scFv) molecules, dAb fragments, and minimal recognition units consisting of the amino acid residues that mimic the hypervariable region of the antibody.

### D. Therapeutic Administration

The methods described herein comprise administering a therapeutic dose of a LIGHT antagonist to a subject.

In accordance with the methods of the present invention, a safe therapeutic dose of a LIGHT antagonist that is administered to a subject will vary depending upon the age and the size (e.g., body weight or body surface area) of the subject, as well as the route of administration, and other factors well known to those of ordinary skill in the art. In certain embodiments, the dose of LIGHT antagonist administered to the subject is from about 20 to about 1200 mg, about 30 to about 1200 mg, about 40 to about 1200 mg, about 120 to about 900 mg, about 300 to about 600 mg, about 20 to about 900 mg, about 30 to about 900 mg, about 40 to about 900 mg, about 20 to about 600 mg, about 30 to about 600 mg, about 40 to about 600 mg, about 20 to about 300 mg, about 30 to about 300, about 40 to about 300 mg, about 20 to about 120 mg, about 30 to about 120 mg, about 40 to about 120 mg, about 120 to about 1200 mg, about 120 to about 900 mg, about 120 to about 600 mg, about 120 to about 300 mg, about 300 to about 1200 mg, about 300 to about 900 mg, about 300 to about 600 mg, about 600 to about 1200 mg, about 600 to about 900 mg, or about 900 to about 1200 mg. In one embodiment, the dose is 1200 mg.

For example, the present invention includes methods wherein about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, about 1000 mg, about 1005 mg, about 1010 mg, about 1015 mg, about 1020 mg, about 1025 mg, about 1030 mg, about 1035 mg, about 1040 mg, about 1045 mg, about 1050 mg, about 1055 mg, about 1060 mg, about 1065 mg, about 1070 mg, about 1075 mg, about 1080 mg, about 1085 mg, about 1090 mg, about 1095 mg, about 1100 mg, about 1105 mg, about 1110 mg, about 1115 mg, about 1120 mg, about 1125 mg, about 1130 mg, about 1135 mg, about 1140 mg, about 1145 mg, about 1150 mg, about 1155 mg, about 1160 mg, about 1165 mg, about 1170 mg, about 1175 mg, about 1180 mg, about 1185 mg, about 1190 mg, about 1195 mg, or about 1200 mg of a LIGHT antagonist is administered to the subject.

In certain exemplary embodiments, the anti-LIGHT antagonist is administered to the subject as a subcutaneous dose. Other exemplary modes of administration include, but are not limited to, intradermal, intramuscular, intraperitoneal, intravenous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The LIGHT antagonist can be administered parenterally or subcutaneously.

Various delivery systems are known and can be used to administer the pharmaceutical composition of the invention, e.g., encapsulation in liposomes, microparticles, microcapsules, receptor mediated endocytosis (see, e.g., Wu et al. (1987) J. Biol. Chem. 262:4429-4432). The therapeutic compositions of the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311. In certain exemplary embodiments, an anti-LIGHT antagonist of the invention is formulated according to the methods disclosed in co-pending application 61/615,539, entitled “STABLE ANTI-LIGHT BINDING AGENT FORMULATIONS”, filed on Mar. 26, 2012, and which is specifically incorporated by reference herein.

Pharmaceutical compositions of the invention may be prepared into dosage forms in a unit dose suited to fit a dose of the active ingredients. Such dosage forms in a unit dose include, for example, tablets, pills, capsules, injections (ampoules), suppositories, etc.

Pharmaceutical compositions can also be administered to the subject using any acceptable device or mechanism. For example, the administration can be accomplished using a syringe and needle or with a reusable pen and/or autoinjector delivery device. The methods of the present invention include the use of numerous reusable pen and/or autoinjector delivery devices to administer a LIGHT antagonist (or pharmaceutical formulation comprising the antagonist). Examples of such devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany), to name only a few. Examples of disposable pen and/or autoinjector delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park, Ill.), to name only a few.

The use of a microinfusor to deliver a LIGHT antagonist (or pharmaceutical formulation comprising the antagonist) to a subject is also contemplated herein. As used herein, the term “microinfusor” means a subcutaneous delivery device designed to slowly administer large volumes (e.g., up to about 2.5 mL or more) of a therapeutic formulation over a prolonged period of time (e.g., about 10, 15, 20, 25, 30 or more minutes). See, e.g., U.S. Pat. No. 6,629,949; U.S. Pat. No. 6,659,982; and Meehan et al., J. Controlled Release 46:107-116 (1996). Microinfusors are particularly useful for the delivery of large doses of therapeutic proteins contained within high concentration (e.g., about 100, 125, 150, 175, 200 or more mg/mL) and/or viscous solutions.

### E. Combination Therapies

In certain aspects, the present invention includes methods of treating inflammatory bowel disease (IBD) that comprise administering to a subject in need of such treatment a LIGHT antagonist in combination with at least one additional therapeutic agent. Examples of additional therapeutic agents that can be administered in combination with a LIGHT antagonist in the practice of the methods of the present invention include, but are not limited to, mesalazine, prednisone, TNF inhibitors, azathioprine (Imuran), methotrexate, 6-mercaptopurine, steroids, and any other compound known to treat, prevent, or ameliorate inflammatory bowel disease in a subject. In the present methods, the additional therapeutic agent(s) can be administered concurrently or sequentially with the LIGHT antagonist. For example, for concurrent administration, a pharmaceutical formulation can be made which contains both a LIGHT antagonist and at least one additional therapeutic agent. The amount of the additional therapeutic agent that is administered in combination with the LIGHT antagonist in the practice of the methods of the present invention can be easily determined using routine methods known and readily available in the art.

### F. Kits and Articles of Manufacture

In certain aspects, the present invention includes kits and articles of manufacture containing the LIGHT antagonist and a container. The container can be any kind of container suitable for sterile packaging of the LIGHT antagonist, e.g. a hermetically sealed container such as a vial, an ampoule or sachette or a pre-filled syringe, and the container can be or any of the devices as herein described (e.g. under B), the container can also be contained in such a device (e.g. an ampoule or cartridge). The container can also be a container comprising the hermetically sealed container which comprises the LIGHT antagonist.

The label or packaging insert as used herein described can be any kind of data carrier suitable for arrangement together with the container (e.g. with the hermetically sealed container comprising the LIGHT antagonist or with the container that comprises the LIGHT antagonist-containing container), e.g. in the inside or on the outside or as part of the container, as suitable, e.g. on the outside of the container comprising the LIGHT antagonist, within or on the outside of the container comprising the LIGHT antagonist-containing container.

The label can e.g. be a chip, bar code, a print or leaflet comprising the intended data or information, e.g. a leaflet comprising a printed statement or a label comprising a bar code or a print (e.g. written information or bar code) on the outside of the hermetically sealed container.

In the context of the present invention, the label comprises information related to possible adverse events or side effects in connection with the administration of the LIGHT antagonist as herein described. In addition, the label can also comprise information related to the (i) composition, formulation, concentration and total amount, identity of active ingredient (s) contained in the article of manufacture, i.e. of the LIGHT antagonist, pharmaceutical composition, or formulation of present invention; (ii) indications, contra-indications of the antibody or LIGHT antagonist, pharmaceutical composition, or formulation of present invention; (iii) instructions for use; (iv) quality information such as information about the lot/batch number LIGHT antagonist, pharmaceutical composition, or formulation of present invention, the manufacturing or assembly site or the expiry or sell-by date (v) information concerning the correct storage or handling of the article of manufacture, of the device for application, or of the LIGHT antagonist, pharmaceutical composition, or formulation of present invention; (vi) information concerning the composition of the buffer(s), diluent(s), reagent(s), excipients, carriers, formulations of LIGHT antagonist, pharmaceutical composition, or formulation of present invention; (vii) a warning concerning possible consequences when applying unsuitable dosage or administration regimens and/or use in contraindicated indications and/or for unsuitable patient populations; or any other suitable information.

### EXAMPLES

### Example 1

### Subcutaneous (SC) Toxicity Study

A two-month subcutaneous (SC) toxicity study with a three-month recovery period was performed in cynomolgus monkeys with a fully human IgG4 anti-LIGHT antibody comprising a heavy chain comprising the amino acid sequence of SEQ ID NO: 7 and a light chain comprising the amino acid sequence of SEQ ID NO: 8, referred to herein as the F19 Antibody. The F19 Antibody was administered once every 14 days to cynomolgus monkeys by SC injection for 2 months (5 injections) to determine the potential toxicity of the F19 Antibody and the reversibility of potential compound effects after 3 months. 20 male and 20 female cynomolgus monkeys (approximately 2 years of age) were included in the study. The animals received vehicle (10 mM citrate buffer pH 5.5, 0.05 mg/mL of polysorbate 20, and 40 mg/mL of mannitol, sterile filtered solution) or the F19 Antibody in the vehicle at 6, 30, or 60 mg/kg/dose on days 1, 15, 29, 43, and 57. The dose volume was 1 mL/kg. There were five animals per sex in each group. Two animals per sex per group were designated as recovery animals and were maintained without drug administration for 3 months after completion of dosing.

The animals were observed at least once daily for mortality and clinical signs (including local tolerance at the injection sites). Body temperature was recorded on the days of 1st, 3rd and 5th injection. Body weight was recorded weekly and food consumption was recorded daily.

Ophthalmological examinations were performed once prior to the start of treatment and at the end of the dosing phase. Electrocardiography recordings, hematology, immunophenotyping (lymphocyte subpopulations: T [CD3+/CD4+, CD3+/CD8+], B and NK cells), coagulation and serum chemistry analyses, pro-inflammatory cytokine (TNFα, IL-8, IL-6, IL-1β) determination and urinalysis were performed prior to the start of treatment, after 1 and 2 months of treatment, and at the end of the 3-month recovery period. Samples were collected for determination of the F19 Antibody, anti-F19 Antibody antibodies, and serum LIGHT levels.

The animals were euthanized and necropsied 1 week after last dosing (end of dosing period) and 3-months after last dosing (end of recovery period). Macroscopic examination of all monkeys was performed, weights of selected organs were recorded, and tissue specimens were preserved. A microscopic examination was performed on designated tissues from all animals.

No mortality and no F19 Antibody-related clinical signs were observed at any dose level. F19 Antibody administration did not cause overt changes in body weights and food consumption. Additionally, there were no test article-related ophthalmology findings. Ventricular premature complexes were observed in 3 animals during electrocardiography evaluation (1 female at 6 mg/kg/dose on Day 59, 1 female at 60 mg/kg/dose on Day 31, and 1 male at 60 mg/kg/dose on Day 57), however attribution to treatment is considered unlikely as these changes occur spontaneously in monkeys and were observed only on one day in each animal without any dose-relationship. Immunohistochemical staining of LIGHT by the F19 Antibody in monkey heart is limited to isolated lymphoid/mononuclear cell infiltrates, indicating that F19 Antibody binding would not be expected to impact heart electrical activity.

No F19 Antibody-related effects were observed in hematology, coagulation or serum chemistry parameters, pro-inflammatory cytokines (TNFα, IL-8, IL-6, IL-1β or urinalysis. Immunophenotyping showed no significant changes on lymphocyte subpopulations.

There were no compound-related organ weight or macroscopic changes. Microscopically, the only F19 Antibody-related finding noted was a minimal increase in incidence and severity of multifocal lymphocytic infiltration around small hypodermal vessels at the sites of SC administration and adjacent inguinal areas at 60 mg/kg/dose in both sexes. This increase was not observed at the end of the recovery period.

Based on these data, the No-Observed Adverse Effect Level (NOAEL) for this study was 60 mg/kg/dose, with corresponding Cₘₐₓ and AUC values of 1620 to 1730 μg/mL and 343 000 to 388 000 μg·h/mL, respectively, at the end of the dosing period.

### Absorption, Distribution, Metabolism and Excretion Data

Following SC administration in monkeys at doses of 6, 30, and 60 mg/kg administered once every 2 weeks, the F19 Antibody was slowly absorbed with the maximum serum concentration observed between 48 to 144 hours. Less than a dose proportional increase in AUC₀₋₃₃₆ was observed over the range of 6- to 0 mg/kg/dose on both Day 15 and 57. A 10-fold increase in dose leads to 7.83 to 8.77-fold increase in AUC₀₋₃₃₆ on Day 57. Repeated F19 Antibody administrations for 2 months resulted in slightly increased exposure of F19 Antibody in both males and females (Day 57/Day 15 AUC₀₋₃₃₆ ratios of 1.01 to 1.82 in males and 1.27 to 1.90 in females). No overt difference in exposure between male and female monkeys. No intravenous (IV) study with the F19 Antibody was conducted, thus the bioavailability could not be calculated.

The steady-state volume of distribution of the F19 Antibody was not determined in the relevant toxicological species, monkey, as no intravenous (IV) study was conducted. No metabolism studies were conducted with the F19 Antibody since it is a monoclonal antibody. It is generally recognized that the metabolism of biotechnology-derived products results in degradation into small peptides and individual amino acids.

Following SC administration in the 2 month toxicology study in monkeys, the F19 Antibody apparent terminal half-lives ranged from 12.7 to 20.1 days. The half-life values were not apparently different at the doses of 6, 30, and 60 mg/kg once every 2 weeks. An excretion balance study for the F19 Antibody was not conducted because of the difficulty in producing a suitable radiolabeled version of the drug and issues with data interpretation.

No exposure was measured in the in vivo xenograft model versus host disease model in mice where efficacy was apparent starting from 1 and 2 mg/kg doses. The NOAEL dose was determined to be 60 mg/kg once every 2 weeks in the 2 month toxicology study in monkeys. The human equivalent dose can also be considered to be 60 mg/kg.

### Example 2

### Randomized, Double-blinded, Placebo-controlled Phase I Study of the Safety, Tolerability, and Pharmacokinetics of Ascending Single Subcutaneous Doses of the F19 Anti-LIGHT Antibody Conducted in Healthy Volunteers

This study is the first investigation of the F19 antibody, an anti-LIGHT antibody, in humans, and involves careful dose escalation in healthy subjects to obtain initial information on the safety, tolerability, and pharmacokinetics (PK) of single subcutaneous (SC) doses. Specifically, this study involved administering a fully human IgG4 anti-LIGHT antibody comprising a heavy chain comprising the amino acid sequence of SEQ ID NO: 7 and a light chain comprising the amino acid sequence of SEQ ID NO: 8 (the F19 Antibody).

The study had three objectives. First, to assess in healthy subjects the tolerability and safety of the F19 Antibody after ascending single subcutaneous (SC) doses. Second, to assess the pharmacokinetic parameters of the F19 Antibody after ascending single SC doses. Third, to assess the immunogenicity of single SC doses of the F19 Antibody. An exploratory objective was to assess serum soluble LIGHT concentrations.

This was a single-center, randomized, double-blind, placebo-controlled, ascending single SC dose study in six sequential cohorts of healthy subjects. Each dose cohort/level consisted of 8 subjects (6 receiving the F19 Antibody and 2 receiving placebo). The design is well established for first-in-human studies and is appropriate to assess safety, tolerability, and preliminary pharmacokinetics (PK).

40 mg was selected as the starting dose. This dose is below the estimated minimum pharmacologically active dose. The maximum dose was 1200 mg (20 mg/kg for a 60 kg subject), which maintains a 3-fold margin of safety relative to the monkey NOAEL of 60 mg/kg.

The dose escalation steps are provided in Table 1. The dose escalation ratio is approximately 3-fold at the first escalation steps and reduces to 1.3 in the last escalation step. This dose escalation regimen is supported by the absence of adverse clinical findings in monkeys.

The following single doses were administered in an ascending manner:

TABLE 1
Dose Levels

| | | Total volume | | |
|---|---|---|---|---|
| | | injected | | Dose |
| Ascending | Dose | Subcutaneously | Number of | Escalation |
| dose level | (mg) | (mL) | injections | Increment |
| 1 | 40 | 0.27 | 1 | — |
| 2 | 120 | 0.8 | 1 | 3 |
| 3 | 300 | 2 | 1 | 2.5 |
| 4 | 600 | 4 | 2 | 2 |
| 5 | 900 | 6 | 3 | 1.5 |
| 6 | 1200 | 8 | 4 | 1.3 |

The maximum SC injection volume was up to 2 mL per injection with up to 4 injections for a single dose administration.

A maximum of 48 healthy male adult subjects between the ages of 18 and 65 were included in 6 dose levels, 8 subjects per dose level, 6 receiving the F19 Antibody and 2 receiving placebo. The demographics and subject characteristics are described further in Table 3, below.

### Study Treatments

The Investigational medicinal product(s) was the F19 Antibody (150 mg/mL) and matching placebo, administered by SC injection in the abdomen. The dose regimen was ascending single doses of 40, 120, 300, 600, 900 and 1200 mg on Day 1 (1 injection for 40 mg, 120 mg, and 300 mg, 2 injections for 600 mg, 3 injections for 900 mg and 4 injections for 1200 mg). Duration of treatment: A single SC dose. Duration of observation: Up to Day 43 (inclusive) for the results reported here (total duration of the study was up to 16 weeks [Day 85±3 days]).

### Criteria for Evaluation: Safety

All of the following parameters were evaluated for analysis until Day 43 (inclusive).
Assessment of adverse events (AE)s/treatment-emergent adverse events
Injection site reaction (ISR) assessments:
   Erythema (diameter and graded severity)
   Edema (diameter and graded severity)
   Present pain assessment (PPI)
Local tolerance
   The injection site reaction assessment, outside the safety
Physical examination and body weight
Clinical laboratory evaluations including hematology, biochemistry, coagulation, urinalysis
Vital signs (blood pressure and heart rate (HR) supine and standing), 12-lead electrocardiogram (ECG)
Immunogenicity of single SC doses of the F19 Antibody (anti-drug antibody [ADA] titers)

### Criteria for Evaluation: Pharmacokinetics:

The following F19 Antibody serum PK parameters were calculated with non-compartmental analysis.

Maximum observed serum concentration (Cₘₐₓ), time to reach maximum serum concentration (tₘₐₓ), Interval between administration time and the sampling time preceding the first concentration above the lower limit of quantification (t_{lag}), area under the serum concentration versus time curve from time zero to the real time corresponding to the last concentration above the lower limit of quantification, tₗₐₛₜ (AUCₗₐₛₜ), area under the serum concentration versus time curve from time zero extrapolated to infinity (AUC), apparent volume of distribution at steady state (Vss/F), apparent total body clearance (CL/F), and terminal half-life (t_{1/2z}).

All available data were used for PK evaluation and only partial serum concentration-time profiles were available at 600 mg (no concentration data available for Days 8, 15, and 85) and 1200 mg (no concentration data available for Days 3, 50, 57, and 85). Consequently, several PK parameters (especially Cₘₐₓ, AUC₀₋₄₂ and AUC for the 600 mg dose and AUCₗₐₛₜ and AUC for the 1200 mg dose) are not reported. Area under the serum concentration versus time curve from time zero to Day 43 (AUC₀₋₄₂) was determined as a supplemental parameter (except for the 600 mg dose level due to missing data for Days 8 and 15, around median Cₘₐₓ).

### Pharmacokinetic Sampling Times and Bioanalytical Methods:

Sampling: Blood samples for PK evaluations were collected at predose (Day 1), and Days 1, 2, 4, 6, 8, 11, 15, 18, 22, 29, 36, 43, 50, 57, and 85 (±1 day for Days 15 to 29; ±2 days for Days 36 to 43; ±3 days for Days 50 to 85) following the F19 Antibody administration.

Assays: Serum concentrations of the F19 Antibody were determined using a validated enzyme-linked immunosorbant assay (ELISA) with a lower limit of quantification (LLOQ) of 313 ng/mL (0.313 mg/L).

### Statistical Methods: Safety

The safety analysis was conducted on all subjects who received the study drug. The on-treatment phase was defined as the time of the first medicinal product (IMP) administration up to Day 43 visit (included). Potentially clinically significant abnormalities (PCSAs) for clinical laboratory test results, vital signs, and ECGs were listed, flagged and summarized for each treatment group (placebo and the F19 Antibody dose levels). Treatment-emergent adverse events (TEAEs) were tabulated (counts and percents) by primary system-organ class, preferred term (PT) and treatment group. Descriptive statistics (summary and time profile plots Mean+/−SD, one curve per treatment group) were generated for each treatment group for selected parameters of interest (ECG, vital signs and laboratory parameters).

For erythema and edema diameter data, raw data and individual peak values were summarized by treatment group, bar chart of mean peak values and histogram of individual peak values were also be provided. Qualitative assessments of PPI, erythema and edema were summarized in frequency tables of number of subjects with the most extreme qualitative assessments. The adverse events are described further in Tables 4-7, below.

### Statistical Methods: Pharmacokinetics:

Pharmacokinetic parameters for the F19 Antibody were summarized for each dose level using descriptive statistics (mean, geometric mean, standard error of the mean [SEM], median, standard deviation [SD], coefficient of variation [CV], minimum, and maximum).

Dose proportionality was assessed using a power model for Cₘₐₓ, AUC₀₋₄₂, AUCₗₐₛₜ, and AUC. The dose effect on t_{1/2}^{z} was assessed with a linear mix model. The distribution of tₘₐₓ values was represented by histogram plots.

### Statistical Methods: Pharmacokinetics

Pharmacokinetic parameters for the F19 Antibody were summarized for each dose level using descriptive statistics (mean, geometric mean, standard error of the mean [SEM], median, standard deviation [SD], coefficient of variation [CV], minimum, and maximum).

Dose proportionality was assessed using a power model for Cₘₐₓ, AUC₀₋₄₂, AUCₗₐₛₜ, and AUC. The dose effect on t_{1/2z} was assessed with a linear mix model. The distribution of tₘₐₓ values was represented by histogram plots.

### Summary of Results

Population characteristics: the study population included 48 healthy subjects (27 male, 21 female) aged between 22 and 64 years.

Safety results: There were no deaths or serious adverse event (SAE) reported in the study. There was 1 adverse event of special interest (AESI) of alanine aminotransferase (ALT) increase (2.8×ULN) on Day 15 in the placebo group. This was related to extensive physical activity and is accompanied by aspartate aminotransferase (AST) and creatinine phosphokinase (CPK) increases at 3.8×ULN and 28.3×ULN at peaks, respectively. This subject also had neutropenea on Day 15.

The majority of TEAEs were of mild intensity and all subjects recovered without sequelae. The most frequently reported TEAE was upper respiratory tract infection (placebo: 1/12, 40 mg: 0/6, 120 mg: 2/6, 300 mg: 1/6, 600 mg: 1/6, 900 mg: 1/6 and 1200 mg: 2/6). All other TEAEs were sporadic among the groups and the overall incidence of TEAEs did not appear to be dose-related. There were few PCSAs in vital signs, laboratory or ECG parameters, which were considered neither clinically significant nor dose related.

On the PPI questionnaire, no pain was reported in placebo, 40, 120 300 and 900 mg groups. There was mild pain reported for 2 injection sites (left and right upper abdomen) by 1 subject and 3 injection sites (left lower, left and right upper abdomen) by 1 subject in the 600 mg and 1200 mg groups respectively.

Erythema was only reported for 2 injection sites (left and right upper abdomen) in the placebo, 600, 900 and 1200 mg groups with 1/12, 2/6, 1/6 and 1/6 subjects respectively. The maximum diameter was found to be 22 mm, 12 mm, 23 mm and 25 mm respectively in the placebo, 600, 900 and 1200 mg groups.

Edema was only observed in placebo (1/12 subjects) and 1200 mg (1/6 subjects) groups only with maximum diameters of 22 mm and 9 mm respectively.

None of the local tolerability issue was considered clinically significant or reported as TEAE. There were slightly higher injection site reactions in the higher dose levels (600 to 1200 mg).

### Pharmacokinetic Results

Pharmacokinetic parameters for serum the F19 Antibody are summarized in Table 2:

TABLE 2
Mean ± SD (geometric mean) [CV %] of serum the F19 Antibody PK parameters

| | the F19 | the F19 | the F19 | the F19 | the F19 | the F19 |
|---|---|---|---|---|---|---|
| PK | Antibody | Antibody | Antibody | Antibody | Antibody | Antibody |
| parameters | 40 mg | 120 mg | 300 mg | 600 mg^{b} | 900 mg^{b} | 1200 mg^{b} |
| N | 6 | 6 | 6 | 6 | 6 | 6 |
| Cₘₐₓ | 5.27 ± 1.53 | 17.9 ± 6.69 | 45.7 ± 16.9 | —^{d} | 122 ± 36.1 | 147 ± 23.8 |
| (mg/L) | (5.11)[29.0] | (17.0)[37.3] | (43.7)[37.1] | | (117)[29.7] | (145)[16.2] |
| tₘₐₓ^{a} | 7.00 | 6.57 | 7.00 | —^{d} | 5.00 | 8.50 |
| (day) | (2.00-28.0) | (2.00-14.0) | (3.00-10.0) | | (2.98-19.0) | (2.00-22.0) |
| t_{lag}^{a} | 0.170 | 0.00 | 0.00 | —^{d} | 0.00 | 0.00 |
| (day) | (0.00-0.500) | (0.00-0.17) | (0.00-0.00) | | (0.00-0.00) | (0.00-0.00) |
| AUC₀₋₄₂ | 148 ± 36.1 | 480 ± 205 | 1270 ± 486 | —^{d} | 3730 ± 976 | 4030 ± 832 |
| (mg · day/L) | (145)[24.4] | (447)[42.7] | (1210)[38.3] | | (3630)[26.2] | (3970)[20.6] |
| AUCₗₐₛₜ | 193 ± 50.1 | 642 ± 267 | 1710 ± 696 | 2430 ± 520 | 4630 ± 1190 | —^{d} |
| (mg · day/L) | (187)[25.9] | (600)[41.6] | (1630)[40.6] | (2380)[21.4] | (4500)[25.7] | |
| AUC | 207 ± 48.3 | 698 ± 298 | 2060 ± 1160 | —^{d} | 4600 ± 1310 | —^{d} |
| (mg · day/L) | (202)[23.3] | (651)[42.7] | (1870)[56.2] | | (4460)[28.5]^{c} | |
| Vₛₛ/F | 6.70 ± 1.70 | 6.83 ± 2.78 | 6.61 ± 1.44 | —^{d} | 5.59 ± 1.58 | —^{d} |
| (L) | (6.50)[25.4] | (6.38)[40.7] | (6.48)[21.7] | | (5.41)[28.3] ^{c} | |
| CL/F | 0.203 ± 0.0530 | 0.196 ± 0.0730 | 0.171 ± 0.0543 | —^{d} | 0.208 ± 0.0576 | —^{d} |
| (L/day) | (0.198)[25.9] | (0.184)[37.4] | (0.160)[31.9] | | (0.202)[27.6] ^{c} | |
| t_{1/2z} | 18.7 ± 3.81 | 21.3 ± 3.23 | 28.9 ± 14.7 | 32.0 ± 14.7 | 13.6 ± 10.2 | —^{d} |
| (day) | (18.3)[20.4] | (21.0)[15.2] | (26.3)[51.0] | (29.3)[45.8] | (11.4)[75.2] | |

^{a}Median (Min-Max);
^{b}Missing or preliminary estimates at 600 to 1200 mg due to partial serum-concentration profiles;
^{c}N = 5, due to area extrapolation >30% in one subject.
^{d}Not reported due to missing data.
Note:
For 40 to 300 mg dose groups, the data were available up to Day 85, for 600 to 900 mg dose groups, the data were available up to Day 57, and for 1200 mg dose group, the data were available up to Day 43.

The F19 Antibody mean t_{1/2z} calculated from available data ranged from 13.6 days to 32 days following single SC doses to healthy subjects. A 22.5-fold increase in dose from 40 mg to 900 mg resulted in 20.1-fold and 21.5 fold increase in geometric mean AUCₗₐₛₜ and AUC, respectively. A 30-fold increase in dose over the entire range of 40 to 1200 mg resulted in a 27.4-fold and 28.6-fold increase in geometric mean Cₘₐₓ and AUC₀₋₄₂, respectively.

### Conclusions: Pharmacokinetics

The F19 Antibody was absorbed with a medium tₘₐₓ ranging from 5.0 to 8.5 days and eliminated with a mean t_{1/2z} ranging from 13.6 days to 32 days following single SC doses to healthy subjects.

F19 Antibody exposure increased in a close to dose proportional manner, with a 30-fold increase over the entire dose range of 40 to 1200 mg resulting in a 27.4-fold and 28.6-fold increase in geometric mean Cₘₐₓ and AUC₀₋₄₂, respectively. Table 7, below, provides further dose proportionality assessments.

### Conclusions: Safety

The F19 Antibody was generally well tolerated. Treatment-emergent adverse events, abnormal vital signs, ECG parameters or laboratory values were relatively infrequent in all groups and there were no apparent dose dependent differences among the treatment groups.

There were no clinically important local tolerability issues in the treatment groups.

TABLE 3
Demographics and subject characteristics at baseline - safety population

| | | | F19 Antibody | | | | | | | | | | | | | |
|---|---|---|---|---|---|---|---|---|---|---|---|---|---|---|---|---|
| | Placebo | | 40 mg | | 120 mg | | 300 mg | | 600 mg | | 900 mg | | 1200 mg | | All | |
| | (N = 12) | | (N = 6) | | (N = 6) | | (N = 6) | | (N = 6) | | (N = 6) | | (N = 6) | | (N = 48) | |
| Age (years) | | | | | | | | | | | | | | | | |
| Number | 12 | | 6 | | 6 | | 6 | | 6 | | 6 | | 6 | | 48 | |
| Mean (SD) | 45.3 | (8.9) | 46.0 | (16.6) | 39.3 | (13.8) | 41.2 | (12.2) | 38.8 | (7.7) | 41.3 | (11.0) | 36.7 | (11.1) | 41.7 | (11.3) |
| Min:Max | 36:64 | | 25:62 | | 22:57 | | 24:56 | | 28:48 | | 29:56 | | 27:58 | | 22:64 | |
| Sex [n (%)] | | | | | | | | | | | | | | | | |
| Number | 12 | | 6 | | 6 | | 6 | | 6 | | 6 | | 6 | | 48 | |
| Male | 7 | (58.3%) | 5 | (83.3%) | 3 | (50.0%) | 3 | (50.0%) | 3 | (50.0%) | 2 | (33.3%) | 4 | (66.7%) | 27 | (56.3%) |
| Female | 5 | (41.7%) | 1 | (16.7%) | 3 | (50.0%) | 3 | (50.0%) | 3 | (50.0%) | 4 | (66.7%) | 2 | (33.3%) | 21 | (43.8%) |
| Race [n (%)] | | | | | | | | | | | | | | | | |
| Number | 12 | | 6 | | 6 | | 6 | | 6 | | 6 | | 6 | | 48 | |
| Caucasian/ | 9 | (75.0%) | 0 | | 6 | (100%) | 6 | (100%) | 6 | (100%) | 5 | (83.3%) | 6 | (100%) | 38 | (79.2%) |
| White | | | | | | | | | | | | | | | | |
| Black | 2 | (16.7%) | 0 | | 0 | | 0 | | 0 | | 1 | (16.7%) | 0 | | 3 | (6.3%) |
| Other | 1 | (8.3%) | 6 | (100%) | 0 | | 0 | | 0 | | 0 | | 0 | | 7 | (14.6%) |
| Weight (kg) | | | | | | | | | | | | | | | | |
| Number | 12 | | 6 | | 6 | | 6 | | 6 | | 6 | | 6 | | 48 | |
| Mean (SD) | 69.75 | (10.83) | 75.45 | (6.86) | 71.62 | (10.53) | 75.15 | (15.78) | 73.88 | (10.23) | 78.10 | (7.40) | 73.68 | (13.48) | 73.42 | (10.71) |
| Min:Max | 50.2:86.3 | | 68.1:86.3 | | 56.3:86.3 | | 50.4:91.6 | | 61.1:86.3 | | 65.9:86.3 | | 59.2:91.4 | | 50.2:91.6 | |
| BMI (kg/m²) | | | | | | | | | | | | | | | | |
| Number | 12 | | 6 | | 6 | | 6 | | 6 | | 6 | | 6 | | 48 | |
| Mean (SD) | 25.06 | (3.02) | 26.14 | (2.24) | 24.98 | (2.33) | 26.00 | (3.62) | 26.09 | (1.95) | 26.81 | (2.01) | 26.45 | (3.20) | 25.82 | (2.65) |

| | | | | | | | | |
|---|---|---|---|---|---|---|---|---|
| Min:Max | 20.1:30.0 | 22.5:29.0 | 22.8:29.1 | 20.8:29.4 | 24.1:29.3 | 24.2:29.9 | 21.7:29.9 | 20.1:30.0 |

TABLE 4
Number (%) of subjects with TEAE(s) by Primary SOC and PT - safety population

| | | F19 Antibody | | | | | |
|---|---|---|---|---|---|---|---|
| Primary system organ class | Placebo | 40 mg | 120 mg | 300 mg | 600 mg | 900 mg | 1200 mg |
| Preferred term [n (%)] | (N = 12) | (N = 6) | (N = 6) | (N = 6) | (N = 6) | (N = 6) | (N = 6) |
| Any class | 5 (41.7%) | 4 (66.7%) | 4 (66.7%) | 2 (33.3%) | 3 (50.0%) | 1 (16.7%) | 2 (33.3%) |
| Infections and infestations | 1 (8.3%) | 1 (16.7%) | 2 (33.3%) | 2 (33.3%) | 1 (16.7%) | 1 (16.7%) | 2 (33.3%) |
| Upper respiratory tract | 1 (8.3%) | 0 | 2 (33.3%) | 1 (16.7%) | 1 (16.7%) | 1 (16.7%) | 2 (33.3%) |
| infection | | | | | | | |
| Gingivitis | 0 | 1 (16.7%) | 0 | 0 | 0 | 0 | 0 |
| Nasopharyngitis | 0 | 0 | 0 | 1 (16.7%) | 0 | 0 | 0 |
| Nervous system disorders | 0 | 1 (16.7%) | 0 | 0 | 0 | 0 | 1 (16.7%) |
| Headache | 0 | 1 (16.7%) | 0 | 0 | 0 | 0 | 1 (16.7%) |
| Gastrointestinal disorders | 2 (16.7%) | 1 (16.7%) | 2 (33.3%) | 0 | 2 (33.3%) | 0 | 0 |
| Diarrhoea | 1 (8.3%) | 1 (16.7%) | 2 (33.3%) | 0 | 1 (16.7%) | 0 | 0 |
| Nausea | 1 (8.3%) | 0 | 0 | 0 | 0 | 0 | 0 |
| Toothache | 0 | 0 | 0 | 0 | 1 (16.7%) | 0 | 0 |
| Vomiting | 1 (8.3%) | 0 | 0 | 0 | 0 | 0 | 0 |
| Skin and subcutaneous tissue | 0 | 1 (16.7%) | 0 | 0 | 1 (16.7%) | 0 | 0 |
| disorders | | | | | | | |
| Dermatitis | 0 | 0 | 0 | 0 | 1 (16.7%) | 0 | 0 |
| Dermatitis contact | 0 | 1 (16.7%) | 0 | 0 | 0 | 0 | 0 |
| Musculoskeletal and | 2 (16.7%) | 1 (16.7%) | 0 | 0 | 0 | 0 | 0 |
| connective tissue disorders | | | | | | | |
| Back pain | 0 | 1 (16.7%) | 0 | 0 | 0 | 0 | 0 |
| Musculoskeletal pain | 1 (8.3%) | 0 | 0 | 0 | 0 | 0 | 0 |
| Pain in extremity | 1 (8.3%) | 0 | 0 | 0 | 0 | 0 | 0 |
| General disorders and | 0 | 1 (16.7%) | 1 (16.7%) | 0 | 0 | 0 | 0 |
| administration site | | | | | | | |
| conditions | | | | | | | |
| Influenza like illness | 0 | 1 (16.7%) | 0 | 0 | 0 | 0 | 0 |
| Injection site pruritus | 0 | 0 | 1 (16.7%) | 0 | 0 | 0 | 0 |
| Investigations | 1 (8.3%) | 0 | 0 | 0 | 0 | 0 | 1 (16.7%) |
| Blood creatine | 1 (8.3%) | 0 | 0 | 0 | 0 | 0 | 1 (16.7%) |
| phosphokinase increased | | | | | | | |
| Alanine aminotransferase | 1 (8.3%) | 0 | 0 | 0 | 0 | 0 | 0 |
| increased | | | | | | | |
| Neutrophil count | 1 (8.3%) | 0 | 0 | 0 | 0 | 0 | 0 |
| decreased | | | | | | | |

TEAE: Treatment emergent adverse event,
SOC: System organ class,
PT: Preferred term MedDRA 15.1
N = Number of subjects treated within each group, n (%) = number and % of subjects with at least one TEAE in each category
Note:
Table sorted by SOC internationally agreed order and decreasing frequency of PT in F19 1200 mg group. An adverse event is considered as treatment emergent if it occurred from the time of the first investigational medicinal product (IMP) administration up to Day 43 (included).

TABLE 5
Erythema diameter data (mm) - Descriptive statistics of individual
peak values until Day 43 - safety population

| | Raw data | | | | | | |
|---|---|---|---|---|---|---|---|
| | N | Mean | SD | SEM | Median | Min | Max |
| Placebo | | | | | | | |
| Right upper abdomen | 6 | 3.7 | 9.0 | 3.67 | 0.0 | 0 | 22 |
| Right lower abdomen | 2 | 0.0 | 0.0 | 0.00 | 0.0 | 0 | 0 |
| Left upper abdomen | 12 | 0.0 | 0.0 | 0.00 | 0.0 | 0 | 0 |
| Left lower abdomen | 4 | 0.0 | 0.0 | 0.00 | 0.0 | 0 | 0 |
| F19 40 mg | | | | | | | |
| Left upper abdomen | 6 | 0.0 | 0.0 | 0.00 | 0.0 | 0 | 0 |
| F19 120 mg | | | | | | | |
| Left upper abdomen | 6 | 0.0 | 0.0 | 0.00 | 0.0 | 0 | 0 |
| F19 300 mg | | | | | | | |
| Left upper abdomen | 6 | 0.0 | 0.0 | 0.00 | 0.0 | 0 | 0 |
| F19 600 mg | | | | | | | |
| Right upper abdomen | 6 | 2.3 | 3.8 | 1.56 | 0.0 | 0 | 9 |
| Left upper abdomen | 6 | 3.5 | 5.5 | 2.25 | 0.0 | 0 | 12 |
| F19 900 mg | | | | | | | |
| Right upper abdomen | 6 | 3.8 | 9.4 | 3.83 | 0.0 | 0 | 23 |
| Left upper abdomen | 6 | 0.0 | 0.0 | 0.00 | 0.0 | 0 | 0 |
| Left lower abdomen | 6 | 0.0 | 0.0 | 0.00 | 0.0 | 0 | 0 |
| F19 1200 mg | | | | | | | |
| Right upper abdomen | 6 | 0.0 | 0.0 | 0.00 | 0.0 | 0 | 0 |
| Right lower abdomen | 6 | 0.0 | 0.0 | 0.00 | 0.0 | 0 | 0 |
| Left upper abdomen | 6 | 4.2 | 10.2 | 4.17 | 0.0 | 0 | 25 |
| Left lower abdomen | 6 | 0.0 | 0.0 | 0.00 | 0.0 | 0 | 0 |

TABLE 6
Edema diameter data (mm) - Descriptive statistics of individual
peak values until Day 43 - safety population

| | Raw data | | | | | | |
|---|---|---|---|---|---|---|---|
| | N | Mean | SD | SEM | Median | Min | Max |
| Placebo | | | | | | | |
| Right upper abdomen | 6 | 3.7 | 9.0 | 3.67 | 0.0 | 0 | 22 |
| Right lower abdomen | 2 | 0.0 | 0.0 | 0.00 | 0.0 | 0 | 0 |
| Left upper abdomen | 12 | 0.0 | 0.0 | 0.00 | 0.0 | 0 | 0 |
| Left lower abdomen | 4 | 0.0 | 0.0 | 0.00 | 0.0 | 0 | 0 |
| F19 40 mg | | | | | | | |
| Left upper abdomen | 6 | 0.0 | 0.0 | 0.00 | 0.0 | 0 | 0 |
| F19 120 mg | | | | | | | |
| Left upper abdomen | 6 | 0.0 | 0.0 | 0.00 | 0.0 | 0 | 0 |
| F19 300 mg | | | | | | | |
| Left upper abdomen | 6 | 0.0 | 0.0 | 0.00 | 0.0 | 0 | 0 |
| F19 600 mg | | | | | | | |
| Right upper abdomen | 6 | 0.0 | 0.0 | 0.00 | 0.0 | 0 | 0 |
| Left upper abdomen | 6 | 0.0 | 0.0 | 0.00 | 0.0 | 0 | 0 |
| F19 900 mg | | | | | | | |
| Right upper abdomen | 6 | 0.0 | 0.0 | 0.00 | 0.0 | 0 | 0 |
| Left upper abdomen | 6 | 0.0 | 0.0 | 0.00 | 0.0 | 0 | 0 |
| Left lower abdomen | 6 | 0.0 | 0.0 | 0.00 | 0.0 | 0 | 0 |
| F19 1200 mg | | | | | | | |
| Right upper abdomen | 6 | 0.0 | 0.0 | 0.00 | 0.0 | 0 | 0 |
| Right lower abdomen | 6 | 0.0 | 0.0 | 0.00 | 0.0 | 0 | 0 |
| Left upper abdomen | 6 | 1.5 | 3.7 | 1.50 | 0.0 | 0 | 9 |
| Left lower abdomen | 6 | 1.0 | 2.4 | 1.00 | 0.0 | 0 | 6 |

TABLE 7
Present pain intensity data - Number of subjects with most extreme value until Day 43 - safety
population

| | | F19 Antibody | | | | | |
|---|---|---|---|---|---|---|---|
| | Placebo | 40 mg | 120 mg | 300 mg | 600 mg | 900 mg | 1200 mg |
| Injection site | (N = 12) | (N = 6) | (N = 6) | (N = 6) | (N = 6) | (N = 6) | (N = 6) |
| Right upper abdomen | 6 (50.0%) | 0 | 0 | 0 | 6 (100%) | 6 (100%) | 6 (100%) |
| No Pain | 6 (50.0%) | 0 | 0 | 0 | 5 (83.3%) | 6 (100%) | 6 (100%) |
| Mild | 0 | 0 | 0 | 0 | 1 (16.7%) | 0 | 0 |
| Right lower abdomen | 2 (16.7%) | 0 | 0 | 0 | 0 | 0 | 6 (100%) |
| No Pain | 2 (16.7%) | 0 | 0 | 0 | 0 | 0 | 5 (83.3%) |
| Mild | 0 | 0 | 0 | 0 | 0 | 0 | 1 (16.7%) |
| Left upper abdomen | 12 (100%) | 6 (100%) | 6 (100%) | 6 (100%) | 6 (100%) | 6 (100%) | 6 (100%) |
| No Pain | 12 (100%) | 6 (100%) | 6 (100%) | 6 (100%) | 5 (83.3%) | 6 (100%) | 5 (83.3%) |
| Mild | 0 | 0 | 0 | 0 | 1 (16.7%) | 0 | 1 (16.7%) |
| Left lower abdomen | 4 (33.3%) | 0 | 0 | 0 | 0 | 6 (100%) | 6 (100%) |
| No Pain | 4 (33.3%) | 0 | 0 | 0 | 0 | 6 (100%) | 5 (83.3%) |
| Mild | 0 | 0 | 0 | 0 | 0 | 0 | 1 (16.7%) |

What is claimed is:

1. A maximal safe therapeutic dose of a LIGHT antagonist which, following administration of a single dose to a human subject, has one or more of the properties selected from the group consisting of:
(a) an area under the plasma concentration versus time curve calculated using the trapezoidal method from time zero to real time (AUCₗₐₛₜ) from about 100 mg·day/L to about 6000 mg·day/L;
(b) an area under the plasma concentration versus time curve extrapolated to infinity (AUC) from about 150 mg·day/L to about 6000 mg·day/L;
(c) a maximum plasma concentration observed (Cₘₐₓ) from about 3.5 mg/L to about 175 mg/L;
(d) a first time to reach a maximum plasma concentration (tₘₐₓ) from about 4 days to about 9 days; and
(e) a time to reach terminal half life (t_{1/2}^{Z}) from about 3 days to about 47 days.

2. The dose of claim 1, wherein the LIGHT antagonist is an antibody or antigen-binding fragment that specifically binds LIGHT.

3. The dose of claim 1, wherein the antibody or antigen-binding fragment comprises:
(a) heavy and light chain CDR sequences from the HCVR/LCVR sequence pair of SEQ ID NOs: 10/11;
(b) three heavy chain complementarity determining region (HCDR) sequences comprising SEQ ID NOs: 1, 2, and 3, respectively, and three light chain complementarity determining (LCDR) sequences comprising SEQ ID NOs: 4, 5, and 6, respectively; or
(c) an HCVR having the amino acid sequence of SEQ ID NO: 10 and an LCVR having the amino acid sequence of SEQ ID NO: 11.

4. The dose of claim 1, wherein the therapeutic dose is equal to or less than about 1200 mg.

5. The dose of claim 1, wherein the therapeutic dose is selected from the group consisting of 40 mg, 120 mg, 300 mg, 600 mg, 900 mg and 1200 mg.

6. The dose of claim 1, wherein the therapeutic dose is 1200 mg.

7. A method of monitoring whether a therapeutic dose of a LIGHT antagonist administered to a human subject is safe, said method comprising:
(a) administering said therapeutic dose of said LIGHT antagonist to said human subject;
(b) measuring one or more events selected from the group consisting of: intensive treatment in an emergency room or at home for allergic bronchospasm, blood dyscrasias, or convulsions; development of drug dependency or drug abuse; alanine aminotransferase (AL T)>3× upper limit of normal range (ULN) associated with total bilirubin >2×ULN or ALT increase >2×ULN; diagnosis of cancer during study; QTc≧=500 ms; systemic hypersensitivity reactions or anaphylaxis; severe injection site reaction; infection including opportunistic infections; pregnancy; and
overdose, and
(c) determining one or more said events as measured in (b) has occurred, wherein said therapeutic dose is identified as not safe and the therapeutic dose is discontinued or lowered.

8. The method of claim 7, wherein the infection is an upper respiratory tract infection.

9. The method of claim 7, wherein the injection site reaction is erythema, pain, or edema.

10. The method of claim 9, wherein the erythema diameter is ≧9 mM.

11. The method of claim 9, wherein the edema diameter is ≧9 mM.

12. The method of claim 9, wherein the pain is assessed as at least mild using the present pain intensity (PPI) assessment.

13. The method of claim 9, wherein the LIGHT antagonist is an antibody or antigen-binding fragment that specifically binds LIGHT.

14. The method of claim 14, wherein the antibody or antigen-binding fragment comprises:
(a) heavy and light chain CDR sequences from the HCVR/LCVR sequence pair of SEQ ID NOs: 10/11;
(b) three heavy chain complementarity determining region (HCDR) sequences comprising SEQ ID NOs: 1, 2, and 3, respectively, and three light chain complementarity determining (LCDR) sequences comprising SEQ ID NOs: 4, 5, and 6, respectively; or
(c) an HCVR having the amino acid sequence of SEQ ID NO: 10 and an LCVR having the amino acid sequence of SEQ ID NO: 11.

15. The method of claim 9, wherein the therapeutic dose is equal to or less than about 1200 mg.

16. The method of claim 9, wherein the therapeutic dose is selected from the group consisting of 40 mg, 120 mg, 300 mg, 600 mg, 900 mg and 1200 mg.

17. The method of claim 9, wherein the therapeutic dose is 1200 mg.

18. A method of monitoring whether a therapeutic dose of a LIGHT antagonist administered to a human subject is safe, said method comprising:
(a) administering said therapeutic dose of said LIGHT antagonist to said human subject;
(b) measuring one or more events selected from the group consisting of: intensive treatment in an emergency room or at home for allergic bronchospasm, blood dyscrasias, or convulsions; development of drug dependency or drug abuse; alanine aminotransferase (AL T)>3× upper limit of normal range (ULN) associated with total bilirubin >2×ULN or ALT increase >2×ULN; diagnosis of cancer during study; QTc≧=500 ms; systemic hypersensitivity reactions or anaphylaxis; severe injection site reaction; infection including opportunistic infections; pregnancy; and
overdose, and
(c) determining one or more said events as measured in (b) has not occurred, wherein said therapeutic dose is identified as said safe therapeutic dose having been administered to said human subject.

19. The method of claim 18, wherein the infection is an upper respiratory tract infection.

20. The method of claim 18, wherein the injection site reaction is erythema, pain, or edema.

21. The method of claim 20, wherein the erythema diameter is ≧9 mM.

22. The method of claim 20, wherein the edema diameter is ≧9 mM.

23. The method of claim 20, wherein the pain is assessed as at least mild using the present pain intensity (PPI) assessment.

24. The method of claim 18, wherein the LIGHT antagonist is an antibody or antigen-binding fragment that specifically binds LIGHT.

25. The method of claim 24, wherein the antibody or antigen-binding fragment comprises:
(a) heavy and light chain CDR sequences from the HCVR/LCVR sequence pair of SEQ ID NOs: 10/11;
(b) three heavy chain complementarity determining region (HCDR) sequences comprising SEQ ID NOs: 1, 2, and 3, respectively, and three light chain complementarity determining (LCDR) sequences comprising SEQ ID NOs: 4, 5, and 6, respectively; or
(c) an HCVR having the amino acid sequence of SEQ ID NO: 10 and an LCVR having the amino acid sequence of SEQ ID NO: 11.

26. The method of claim 18, wherein the therapeutic dose is equal to or less than about 1200 mg.

27. The method of claim 18, wherein the therapeutic dose is selected from the group consisting of 40 mg, 120 mg, 300 mg, 600 mg, 900 mg and 1200 mg.

28. The method of claim 18, wherein the therapeutic dose is 1200 mg.

29. A method of quantifying or monitoring an amount of anti-drug antibodies in blood serum of a human subject following administration of drug wherein the drug is a LIGHT antagonist, said method comprising:
(a) administering a dose of said LIGHT antagonist to said human subject;
(b) obtaining a sample of said blood serum from said human subject; and
(b) determining the amount of anti-drug antibodies in said serum sample.

30. The method of claim 29, wherein the LIGHT antagonist is an antibody or antigen-binding fragment that specifically binds LIGHT.

31. The method of claim 30, wherein the antibody or antigen-binding fragment comprises:
(a) heavy and light chain CDR sequences from the HCVR/LCVR sequence pair of SEQ ID NOs: 10/11;
(b) three heavy chain complementarity determining region (HCDR) sequences comprising SEQ ID NOs: 1, 2, and 3, respectively, and three light chain complementarity determining (LCDR) sequences comprising SEQ ID NOs: 4, 5, and 6, respectively; or
(c) an HCVR having the amino acid sequence of SEQ ID NO: 10 and an LCVR having the amino acid sequence of SEQ ID NO: 11.

32. The method of claim 30, wherein the dose is equal to or less than about 1200 mg.

33. The method of claim 30, wherein the dose is selected from the group consisting of 40 mg, 120 mg, 300 mg, 600 mg, 900 mg and 1200 mg.

34. The method of claim 30, wherein the dose is 1200 mg.

35. A pharmaceutical composition comprising the therapeutic dose of LIGHT antagonist of claim 1 together with one or more physiologically acceptable excipients.

36. A method for treating an inflammatory bowel disease (IBD) in a subject in need thereof comprising administering to the subject the pharmaceutical composition of claim 35, wherein the LIGHT antagonist is an anti-LIGHT antibody that is administered at a dose of at least 40 mg to about 1200, thereby treating the IBD in the subject.

37. The method of claim 36, wherein the antibody is administered at a dose of about 1200 mg.

37. The method of claim 36, wherein the antibody is a fully human antibody comprising:
(a) heavy and light chain CDR sequences from the HCVR/LCVR sequence pair of SEQ ID NOs: 10/11;
(b) three heavy chain complementarity determining region (HCDR) sequences comprising SEQ ID NOs: 1, 2, and 3, respectively, and three light chain complementarity determining (LCDR) sequences comprising SEQ ID NOs: 4, 5, and 6, respectively; or
(c) an HCVR having the amino acid sequence of SEQ ID NO: 10 and an LCVR having the amino acid sequence of SEQ ID NO: 11.

38. The method of claim 36, wherein the antibody is administered as a single dose.

39. The method of claim 36, wherein the antibody is administered by injection.

40. The method of claim 36, wherein the antibody is administered subcutaneously.

41. A kit comprising the pharmaceutical composition of claim 35 together with a container.

42. The kit of claim 41 further comprising a label.

43. The kit of claim 42, wherein the label contains reference to one or more adverse reactions or side effects in connection with the administration of the LIGHT antagonist.

44. The kit of claim 43, wherein the one or more adverse reactions or side effects are selected from the group consisting of: intensive treatment in an emergency room or at home for allergic bronchospasm, blood dyscrasias, or convulsions; development of drug dependency or drug abuse; alanine aminotransferase (AL T)>3× upper limit of normal range (ULN) associated with total bilirubin >2×ULN or ALT increase >2×ULN; diagnosis of cancer during study; QTc≧=500 ms; systemic hypersensitivity reactions or anaphylaxis; severe injection site reaction; infection including opportunistic infections; pregnancy; and overdose.

45. The kit of claim 44, wherein the injection site reactions are selected from the group consisting of pain, erythema, and edema.

46. The kit of claim 43, wherein the label indicates that the adverse reactions or side effects can primarily occur in higher dose levels.

47. The kit of claim 46, wherein the higher dose levels are about 500 mg or more per injection.

48. The kit of claim 41, wherein the LIGHT antagonist is an antibody or antigen-binding fragment that specifically binds LIGHT.

49. The kit of claim 48, wherein the LIGHT antagonist is antibody is a fully human antibody comprising:
(a) heavy and light chain CDR sequences from the HCVR/LCVR sequence pair of SEQ ID NOs: 10/11;
(b) three heavy chain complementarity determining region (HCDR) sequences comprising SEQ ID NOs: 1, 2, and 3, respectively, and three light chain complementarity determining (LCDR) sequences comprising SEQ ID NOs: 4, 5, and 6, respectively; or
(c) an HCVR having the amino acid sequence of SEQ ID NO: 10 and an LCVR having the amino acid sequence of SEQ ID NO: 11.
